(19) United States
(12) Patent Application Publication
(10) Pub. No.: US 2025/0312505 A1
(43) Pub. Date: Oct. 9, 2025

(54) SCENT DIFFUSERS AND SYSTEMS THEREFOR

(71) Applicant: FLOWGREENS LIMITED, Melton Mowbray (GB)

(72) Inventors: Wen Nivala, Leicester (GB); Christopher Salvidge, Melton Mowbray (GB); Daniel Hartshorn, London (GB)

(21) Appl. No.: 19/170,986

(22) Filed: Apr. 4, 2025

Related U.S. Application Data
(60) Provisional application No. 63/575,491, filed on Apr. 5, 2024.

(51) Int. Cl. A61L 9/14; A61L 9/12

(52) U.S. Cl. CPC A61L 9/14; A61L 9/12; A61L 2209/111; A61L 2209/133; A61L 2209/134

(57) ABSTRACT

A diffuser includes a base liquid tank and a receptacle that receives an oil container. A base liquid valve dispenses base liquid from the base liquid tank to a mixing chamber and an oil valve dispenses oil from the oil container to the mixing chamber. An atomizer of the diffuser atomizes liquid in the mixing chamber to an exterior of the diffuser. In one aspect, the base liquid valve and the oil valve dispense liquid while the atomizer operates. In another aspect, the atomizer atomizes liquid in a direction substantially perpendicular to a gravitational direction that acts on the diffuser when the diffuser is in an operating orientation. In another aspect, the atomizer includes a mesh component at least partially surrounded by a piezoelectric element that vibrates the mesh component.

### CROSS-REFERENCE TO RELATED APPLICATION

This application claims the benefit of, and priority to, U.S. Provisional Application No. 63/575,491, filed on Apr. 5, 2024, and titled “SCENT DIFFUSERS AND REFILL SYSTEMS” (Attorney Docket No. FLG-00100P), which is hereby incorporated by reference in its entirety.

### BACKGROUND

Scent diffusers, such as ultrasonic oil diffusers, typically require the user to add a few drops of fragrant oil, such as an essential oil, to a water reservoir. When activated, a small piezoelectric disk vibrates at an ultrasonic frequency that disturbs the surface of the water where the oil floats to disperse a fragrant mist from the diffuser.

However, in many cases, the fragrant oil evaporates from the surface of the water after an initial period of use so that the diffused scent may be strong initially but has very little scent after the initial period of operation. In addition, conventional ultrasonic oil diffusers do not perform well in small spaces when used for a short period of time, such as in a shower, or in situations where the user is close to the diffuser, because conventional diffusers are typically designed to run for an extended period of time, such as hours, in a larger space.

Users of conventional oil diffusers may struggle with how much oil to add, and at what times, to reach a desired scent strength without wasting oil or making too weak of a solution, such as after many hours of operation or between uses. Many scent diffusers require the user to add a measured amount of essential oil, such as a certain number of drops, to water. The user does not have much control of the strength of the fragrance due to the imprecise mixing and diffusing of the oil. In addition to the inconvenience of having to add drops of oil to a water reservoir, the user may accidentally add the wrong amount of oil or may spill the oil or water.

In some conventional systems, essential oils are premixed with solvents before being sprayed, either to control the concentration of the liquid or to prevent damage to ultrasonic components. However, this approach compromises the essential oils' natural properties and therapeutic integrity, which typically results in a less fresh and less potent scent delivery.

Other types of diffusers, such as diffusers that use a fan to diffuse fragrance, suffer similar problems as those noted above for conventional ultrasonic oil diffusers. In addition, the sound of the fan can create a constant noise that is not conducive to relaxation, especially in smaller spaces or when the user is close to the diffuser.

In addition, user interaction is lacking for conventional diffusers. Information on user fragrance preferences, personalized suggestions for different fragrances, and connectivity to health applications and/or scent refill ordering options are not integrated into current diffusers. Interactive features for lighting, sound, and user interaction for health, wellness, or mindfulness applications are also missing from current diffusers.

Conventional oil diffusers can also take time and attention from the user to clean, such as cleaning the water reservoir, blocked tubes, or hard-to-clean wicks. The amount of cleaning needed can also vary based on the user's water quality and/or use of the diffuser without the user knowing a specific cleaning recommendation based on their use and/or water quality. Not cleaning the diffuser or its components can eventually cause damage to the diffuser and reduce its effectiveness.

### BRIEF DESCRIPTION OF THE DRAWINGS

The features and advantages of the embodiments of the present disclosure will become more apparent from the detailed description set forth below when taken in conjunction with the drawings. The drawings and the associated descriptions are provided to illustrate embodiments of the disclosure and not to limit the scope of what is claimed.

FIG. 1 illustrates a diffuser in a shower according to one or more embodiments.

FIG. 2 is a closer view of the diffuser of FIG. 1 according to one or more embodiments.

FIG. 3 is a front view of a diffuser according to one or more embodiments.

FIG. 4 is a rear view of a diffuser according to one or more embodiments.

FIG. 5 is a bottom perspective view of a diffuser according to one or more embodiments.

FIG. 6 is a side view of a diffuser according to one or more embodiments.

FIG. 7 is a front view of a diffuser with a faceplate removed according to one or more embodiments.

FIG. 8 is a close-up perspective view of an atomizer of a diffuser according to one or more embodiments.

FIG. 9 is a perspective view of a base liquid tank of a diffuser according to one or more embodiments.

FIG. 10 is a front view of the base liquid tank including an atomizer according to one or more embodiments.

FIG. 11 is a side view of a base liquid tank according to one or more embodiments.

FIG. 12 is a translucent top perspective view of a base liquid tank including a base liquid connector and a receptacle for receiving an oil container according to one or more embodiments.

FIG. 13 is a close-up view of a receptacle for an oil container according to one or more embodiments.

FIG. 14 is a translucent close-up view of a receptacle illustrating internal components of the receptacle and its oil valve according to one or more embodiments.

FIG. 15 is a front view of an actuator according to one or more embodiments.

FIG. 16 is a perspective view of an actuator according to one or more embodiments.

FIG. 17 is a transparent front view of a base liquid tank illustrating internal components of the base liquid tank including an actuator that contacts an oil valve and a base liquid valve according to one or more embodiments.

FIG. 18 illustrates the actuation of the actuator of FIG. 17 between contacting the oil valve and contacting the base liquid valve according to one or more embodiments.

FIG. 19 is a transparent bottom perspective view of a base liquid tank illustrating an internal arrangement of the actuator and a motor assembly according to one or more embodiments.

FIG. 20 is a perspective view of the actuator and motor assembly of FIG. 19 shown in isolation.

FIG. 21 illustrates a network environment including diffusers, remote devices, and a server according to one or more embodiments.

FIG. 22 illustrates an example device status graphical user interface displayed on a remote device according to one or more embodiments.

FIG. 23 illustrates an example scent information graphical user interface displayed on a remote device according to one or more embodiments.

FIG. 24 illustrates an example session details graphical user interface displayed on a remote device according to one or more embodiments.

FIG. 25 illustrates an example session history graphical user interface displayed on a remote device according to one or more embodiments.

FIG. 26 illustrates an example device settings graphical user interface displayed on a remote device according to one or more embodiments.

FIG. 27 illustrates an example user settings graphical user interface displayed on a remote device according to one or more embodiments.

FIG. 28 is a flowchart for a controlled mixing process according to one or more embodiments.

FIG. 29 is a flowchart for a controlled amount calculation process according to one or more embodiments.

FIG. 30 is a flowchart for an operating cycle process according to one or more embodiments.

FIG. 31 is a flowchart for an operating cycle process according to one or more embodiments.

FIG. 32 is a flowchart for a diffusion distance control process according to one or more embodiments.

FIG. 33 is a flowchart for an ongoing valve control process according to one or more embodiments.

FIG. 34 is a flowchart for a rinse process according to one or more embodiments.

FIG. 35 is a flowchart for a remaining liquid determination process according to one or more embodiments.

FIG. 36 is a flowchart for a diffuser control process according to one or more embodiments.

FIG. 37 is a flowchart for a diffuser state or condition indication process using one or more lights of the diffuser according to one or more embodiments.

FIG. 38 is a flowchart for an oil container information collection process according to one or more embodiments.

FIG. 39 is a flowchart for a diffuser control process via a remote device according to one or more embodiments.

FIG. 40 is a flowchart for an oil recommendation process according to one or more embodiments.

FIG. 41 is a flowchart for a user setting communication process according to one or more embodiments.

### DETAILED DESCRIPTION

In the following detailed description, numerous specific details are set forth to provide a full understanding of the present disclosure. It will be apparent, however, to one of ordinary skill in the art that the various embodiments disclosed may be practiced without some of these specific details. In other instances, well-known structures and techniques have not been shown in detail to avoid unnecessarily obscuring the various embodiments.

### Example Diffusers

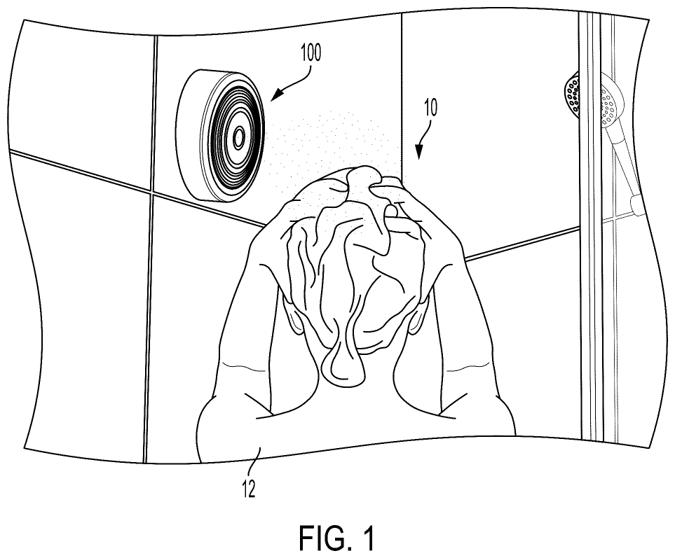

FIG. 1 depicts diffuser 100 in shower 10 according to one or more embodiments. As shown in FIG. 1, diffuser 100 diffuses a fragrant mist or vapor into shower 10 in close proximity to user 12. The fragrant mist or vapor is quickly perceived by user 12 and can rapidly diffuse throughout shower 10.

Diffuser 100 in the example of FIG. 1 is waterproof with its electronics being sealed from water, liquids, and vapor or mist from within diffuser 100 and from outside the diffuser. In some implementations, diffuser 100 is battery powered, such that it does not require external power to operate. In addition, the water or other base liquid is contained within diffuser 100, such that the diffuser does not require an external plumbing connection to a water supply.

In some implementations, diffuser 100 can be mounted onto a wall of shower 10 close to a head-height of user 12. Diffuser 100 may be removably mounted on the wall of shower 10 so that a user can add an oil container and water or another base liquid to the diffuser. In addition, diffuser 100 may also be removably mounted to the wall of shower 10 to facilitate charging the battery of diffuser 100. In some implementations, diffuser 100 may be compatible with or include a stand that allows the diffuser to be portable or taken to other locations, such as being propped up in the operating orientation upon a desk or a nightstand.

The orientation of diffuser 100 shown in FIG. 1 is referred to herein as an operating orientation. As shown in FIG. 1, diffuser 100 differs from conventional diffusers in that the fragrant mist or vapor is diffused in a horizontal direction toward user 12, as opposed to diffusing the fragrant mist or vapor in a vertical direction. As discussed in more detail below, diffuser 100 diffuses a mixture of oil and base liquid, such as water or a type of hydrosol, in a direction that is substantially perpendicular to a gravitational direction in which gravity acts on diffuser 100 when it is in an operating orientation. This can provide a denser fragrant mist or vapor to more directly reach the user's head for a faster and stronger perceived scent, as compared to conventional diffusers that diffuse mist or vapor from a much lower position in a vertical direction against gravity. In this regard, diffuser 100 may be mounted slightly above the user's head to allow gravity to bring the diffused mist or vapor down toward the user's head.

Those of ordinary skill in the art will appreciate with reference to the present disclosure that other implementations of diffuser 100 are possible. For example, diffuser 100 may be used in locations other than a shower, such as in an office, bedroom, or in a commercial space, such as a store.

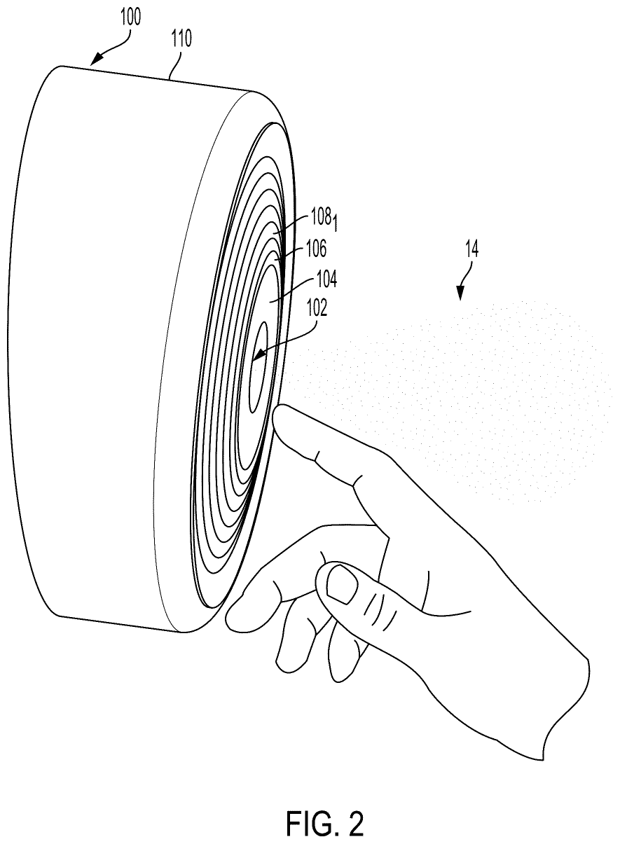

FIG. 2 is a closer view of diffuser 100 according to one or more embodiments. As shown in FIG. 2, diffuser 100 includes exterior shell 110, removable faceplate 1081, lighting ring 106, central button 104, and atomizer 102. Exterior shell 110 provides an outer enclosure of internal components of diffuser 100. In this regard, exterior shell may form a seal with edges of other components, such as removable faceplate 1081 to keep unwanted moisture from entering diffuser 100.

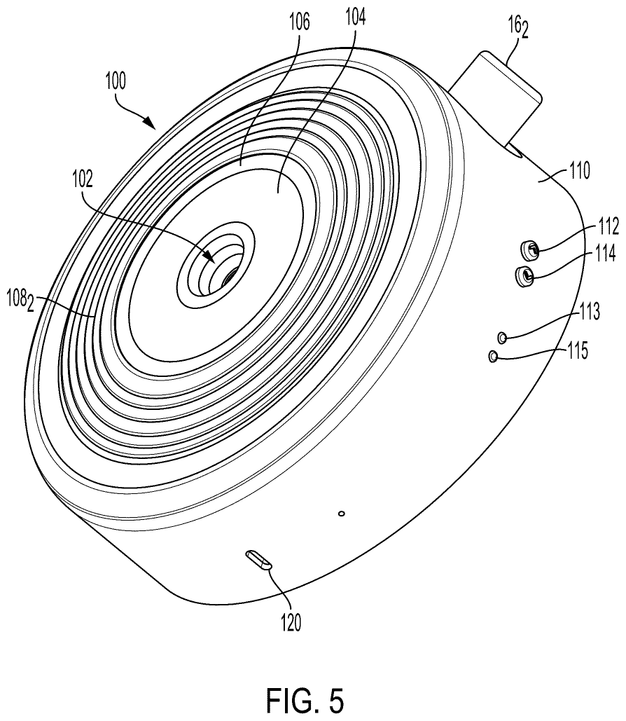

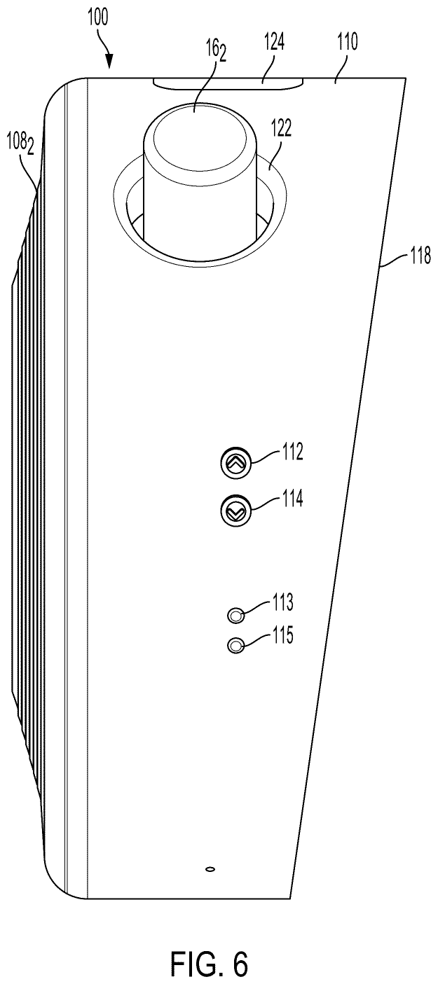

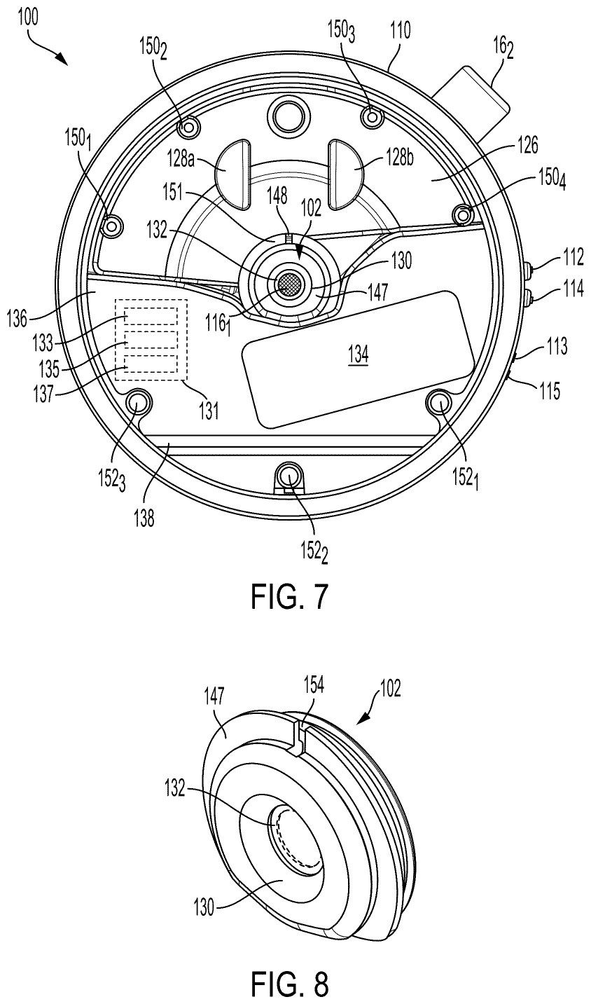

Removable faceplate 1081 is located on a face side of diffuser 100 from which mist or vapor 14 is diffused, and as discussed in more detail below, can provide a way to access internal components of diffuser 100, such as a removable base liquid tank (e.g., base liquid tank 126 in FIG. 7), a battery enclosure (e.g., battery enclosure or compartment 134 in FIG. 7), an electronics enclosure (e.g., electronics enclosure 136 in FIG. 7), and a removable atomizer (e.g., atomizer 102). In some implementations, removable faceplate 1081 is interchangeable with one or more other removable face plates (e.g., faceplate 1082 in FIGS. 5 and 6) that have different appearances from each other and/or different lighting arrangements from each other. The difference in appearance can include, for example, different colors, patterns, and/or materials to provide a customizable appearance to diffuser 100. The difference in lighting arrangements can include, for example, a different quantity and/or placement of lighting elements (e.g., Light Emitting Diodes (LEDs)), directional focus of lighting elements, and/or colors of lighting elements.

Unlike conventional diffusers, diffuser 100 differs in that mist or vapor 14 can be diffused more directly toward the user due to its operating orientation, resulting in a more horizontal diffusion pattern. As noted above, the diffusion of mist or vapor 14 in a direction that is substantially perpendicular to a gravitational direction in which gravity acts on the diffuser when in the operating orientation can facilitate a more targeted dispersion toward a user's head, where the diffused scent can be more quickly perceived by the user with greater strength.

As shown in the side view of FIG. 2, the front of atomizer 102 that diffuses mist or vapor 14 is slightly angled such that the top of exterior shell 110 is wider than the bottom of exterior shell 110. This results in a slightly angled dispersion of mist or vapor 14, which may be taken into consideration when mounting diffuser 100 onto a wall or onto a separate stand. By slightly angling a center of atomizer 102 (e.g., downwards within twenty degrees of horizontal), the dispersion pattern of mist or vapor 14 may be even more directed toward a user's nose in an enclosed space, such as a shower. However, some implementations may not slightly angle the center of atomizer 102 when in the operating orientation.

Central button 104 can provide a user interface of diffuser 100. In some implementations, pressing central button 104 can power on or power off diffuser 100. In other implementations, pressing central button 104 can provide other user input, such as confirming a lighting scheme of light ring 106 or initiating a wireless pairing mode with a remote device (e.g., via Bluetooth) or a joining a network (e.g., Wi-Fi). In this regard, different types of presses on central button 104 may provide different types of user input in some implementations, such as by using a long press, short press, or double press of central button 104.

Lighting ring 106 can provide an indication of a condition or state of diffuser 100, such as the performance of a diffusion operating cycle, a battery charge level of a battery of the diffuser, an oil level of an oil container attached to the diffuser, a base liquid level of a base liquid tank of the diffuser, and/or a type of oil being used by the diffuser. In some implementations, lighting ring 106 can interact with a user, such as by providing breathing guidance for a user, which may interface with a health application, wellness application, or mindfulness application (e.g., a meditation application) executed by a remote device (e.g., a smartphone or tablet) in wireless communication with diffuser 100. As another example, lighting ring 106 may provide chromotherapy or may otherwise be associated with particular moods or goals of the user, which may be obtained via a health application or mindfulness application.

Lighting ring 106 can include lighting elements, such as LEDs, spaced along the circumference of an interior of lighting ring 106. In some implementations, the lighting elements of lighting ring 106 may face a rear side of diffuser 100 or in an alternate direction and provide a backlighting effect of the ring for a more solid or continuous light effect around lighting ring 106. In this regard, lighting ring 106 may form part of removable faceplate 1081, such that changing face plates can include changing the lighting arrangement of diffuser 100, such as by changing lighting elements or the color, filtering, focus, or reflection of light emitted from such lighting elements. In such implementations, one or more electrical contacts of diffuser 100 may provide one or more electrical connections to circuitry of diffuser 100 (e.g., circuitry 131 shown in FIG. 7) for powering and controlling lighting ring 106.

Those of ordinary skill in the art will appreciate with reference to the present disclosure that other implementations of diffuser 100 may differ from the example shown in FIG. 2. For example, diffuser 100 may include a different design of faceplate 1081, may not include lighting ring 106, or may have a more rectangular outer shape than a round outer shape.

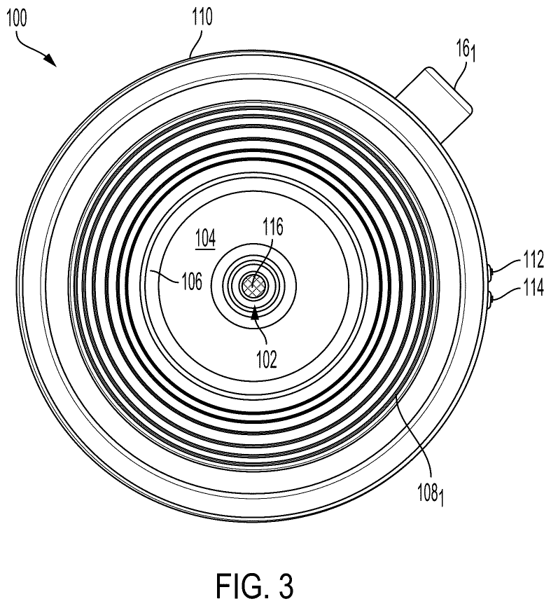

FIG. 3 is a front view of diffuser 100 according to one or more embodiments. As shown in the example of FIG. 3, diffuser 100 includes side buttons 112 and 114, which can be used to increase or decrease a fragrance or scent strength or perform other control operations of diffuser 100, such as to add more time to a diffusion operating cycle or to prematurely end a diffusion operating cycle. In controlling a scent strength, input received via side buttons 112 and 114 can correspond to an amount or of oil dispensed from an oil valve of diffuser 100 or a ratio of oil to base liquid (i.e., a concentration of oil). In some implementations, input received via side buttons 112 and 114 can be confirmed using lighting ring 106. Side buttons 112 and 114 connect to circuitry of diffuser 100 (e.g., circuitry 131 shown in FIG. 7) that executes a firmware of the diffuser to control actuation of at least one actuator for dispensing a controlled amount of oil and/or base liquid.

As noted above, central button 104 may be used to power on or power off diffuser 100 and/or provide user input for other operations. In this regard, an interior surface of central button 104 may actuate or contact a switch of circuitry of diffuser 100 behind faceplate 1081 (e.g., central button contact 151 in FIG. 7). In some implementations, central button 104 may form part of removable faceplate 1081.

Atomizer 102 includes mesh component 116 that is at least partially surrounded by a piezoelectric element that vibrates mesh component 116 to diffuse atomized liquid from atomizer 102. As discussed in more detail below, mesh component 116 may be removable for replacement, cleaning, and/or exchanging with a different mesh component that may have different properties for diffusing an atomized mixture of the base liquid and oil in different diffusion ranges, distances, or patterns. The varying mesh component properties include, for example, the quantity, size, and/or arrangement of holes (e.g., the size of a surface area of mesh component 116 that encompasses holes), a material stiffness of the mesh component, and/or a resonant frequency of the mesh component, which may affect how the mech component vibrates for different signals provided to the piezoelectric element. The different properties of the mesh components can enable the tuning of the diffusion range through power modulation and interaction with the mesh component properties. Accordingly, a first mesh component may vary in material stiffness an/or the quantity, size, and/or arrangement of holes from a second mesh component to vary a diffusion range or distance for an atomized mixture of base liquid and oil.

In some implementations, mesh component 116 may include an electronically readable identifier, such as a Radio Frequency Identification (RFID) tag, Near Field Communication (NFC) tag, barcode, or Quick Response (QR) code that may allow circuitry of the diffuser or an application executed by a remote device (e.g., a smartphone) to identify the mesh component used or to be used in the atomizer. In response to an input indicating use of the mesh component, such as via a radio or optical sensor of the diffuser or via wireless communication with the remote device, the circuitry of the diffuser may adjust the operation of the diffuser, such as by adjusting a signal sent to the piezoelectric element for a target diffusion distance, by adjusting a concentration of oil added to the mixture, or by adjusting a duration for a rinse cycle for a particular hole pattern or material stiffness of the mesh component. In such implementations, a memory of the diffuser's circuitry may store different settings that associate different mesh components, or the application executed by the remote device may provide the input to the diffuser with the setting(s) associated with mesh component being used.

As shown in FIG. 3, an exterior surface of mesh component 116 forms part of an exterior surface of diffuser 100 from which the mist or vapor is diffused directly to air outside of diffuser 100. In some implementations, an interior surface of mesh component 116 forms part of a mixing chamber where the oil and base liquid mix or interact behind atomizer 102. As used herein, references to “mixing” or “mixture” can include heterogenous or homogeneous mixing of liquids, such as where oil heterogeneously mixes with water.

Oil container or bottle 161 can include, for example, an essential oil bottle that may be supplied by the user, by an essential oil manufacturer, or by the manufacturer of diffuser 100. As discussed in more detail below, diffuser 100 includes a receptacle for removably securing or exchanging one or more oil containers for containing a scented oil. In some implementations, the receptacle can include threading for one or more standard threads used for the oil containers. In such implementations, the user may screw oil container 161 into the receptacle of diffuser 100.

An oil valve of diffuser 100 can control the amount of oil dispensed from oil container 161 due to the inverted positioning of oil container 161 with respect to a gravitational force that acts upon diffuser 100 when in the operating orientation. In some implementations, diffuser 100 may be removable from a holding structure or stand used to keep diffuser 100 in an upright position or in the operating orientation so that a user can remove or insert an oil container, add base liquid to a base liquid tank of the diffuser, remove or change mesh component 116, remove or change removable faceplate 1081, and/or otherwise clean or transport diffuser 100. In this regard, diffuser 100 can be portable and is not required to be fixed to a particular location, such as the shower wall discussed above for FIGS. 1 and 2.

Those of ordinary skill in the art will appreciate with reference to the present disclosure that other arrangements of diffuser 100 are possible, such as different locations or styles for side buttons 112 and 114, or the particular location of oil container 161 when inserted into diffuser 100. For example, in other implementations, oil container 161 may instead be inserted on an opposite side of exterior shell 110 or may be inserted at a top portion of exterior shell 110.

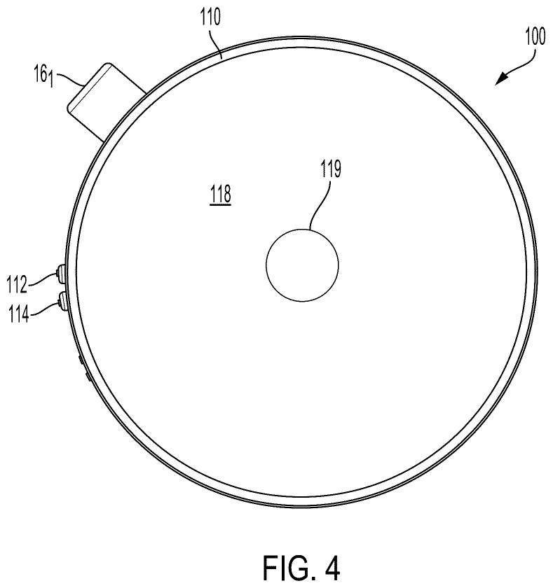

FIG. 4 is a rear view of diffuser 100 according to one or more embodiments. As shown in FIG. 4, diffuser 100 includes rear surface 118, which may be a flat surface in some implementations to facilitate mounting on a wall. In the example of FIG. 4, rear surface 118 includes attachment component 119 configured to removably attach or removably mount diffuser 100 onto a holder (not shown) that may be fixed onto a wall, or other surface, or may form part of a stand, for example. In some implementations, diffuser 100 can be lifted off of the holder by a user and taken to a different location or attached onto a holder without using tools. In some implementations, attachment component 119 can be omitted or may form a different configuration, such as a rectangular lip across rear surface 118 for mounting diffuser 100 onto a wall or stand.

FIG. 5 is a bottom perspective view of diffuser 100 according to one or more embodiments. As shown in FIG. 5, diffuser 100 includes removable faceplate 1082, which differs in appearance from removable faceplate 1081 shown in FIGS. 2 and 3. For example, the color, material, and/or surface texture pattern of faceplate 1081 may differ from that of faceplate 1082. This can facilitate a degree of customization or personalization of diffuser 100, such as to better fit into a color scheme used for a particular shower or room. In some implementations, light ring 106 may form part of removable faceplate 1082.

The example of FIG. 5 also includes oil container or bottle 162, which differs from oil container 161 shown in FIGS. 3 and 4. As discussed above, diffuser 100 includes a receptacle for removably receiving or exchanging one or more oil containers for containing a scented oil. In some implementations, the receptacle can include threading for one or more standard threads used for the oil containers. The user may screw oil container 162 into diffuser 100 and an oil valve of diffuser 100 can control the amount of oil dispensed from oil container 162 due to the inverted positioning of oil container 162 with respect to a gravitational force that acts upon diffuser 100 when in the operating orientation.

In some implementations, the receptacle of diffuser 100 may be removable from the diffuser to facilitate attachment of the oil container into the receptacle without inverting the oil container. The assembly of the oil container and the receptacle may then be inserted into the diffuser in an inverted orientation, while the receptacle provides a seal for containing the oil in the oil container.

As shown on the side of exterior shell 110, side lights 113 and 115 can be used to notify a user, such as to provide information on an operating cycle, a rinse cycle, or other status or condition of the diffuser. For example, side lights 113 and 115 can respond to user input received from buttons 112 and 114, respectively, to indicate to the user that their user input to increase or decrease a scent strength or diffusion distance have been received. As another example, one or both of side lights 113 and 115 may illuminate red to indicate an error condition (e.g., a low oil level, low base liquid level, or low battery charge) or a final minute of a preset operating cycle. In some implementations, side light 113 and/or side light 115 may be used for cases where the diffuser does not include lighting ring 106, or vice versa. Side light 113 and side light 115 can include, for example, LEDs.

Connection 120 shown in FIG. 5 can provide for, in some implementations, charging a battery of diffuser 100, or otherwise powering diffuser 100. Alternatively or additionally, connection 120 can provide an interface with circuitry of diffuser 100 for a remote device, such as a computer, smartphone, or tablet. In some implementations, connection 120 can include a Universal Serial Bus (USB) connection, for example.

FIG. 6 is a side view of diffuser 100 according to one or more embodiments. As shown in FIG. 6, diffuser 100 includes shell aperture 122 in exterior shell 110 and shell opening 124 at a top portion of exterior shell 110. Shell aperture 122 can enable a user to remove and insert oil containers, such as oil container 162, or receptacles that have been secured to an oil containers. Shell opening 124 can enable a user to add a base liquid, such as water, to an internal base liquid tank (e.g., base liquid tank 126 in FIG. 7).

In addition, the side view of diffuser 100 shows that rear surface 118 is slanted or angled with respect to a gravitational direction acting upon diffuser 100 in the example of FIG. 6. This angle or slant can provide for a slightly angled dispersion of an atomized mixture of base liquid and oil when diffuser 100 is mounted to a flat wall. In other cases, the diffuser may be mounted to an adjustable stand that can enable a user to adjust the angle of dispersion. As discussed above with reference to FIG. 1, an angled dispersion (e.g., downwards or upwards towards a user's head) can provide for a more targeted diffusion of mist or vapor towards a user's face. Such an angled diffusion can help keep most of the atomized mixture in the vicinity of the user's head, as opposed to having some of the atomized mixture being diffused upwards and away from the user's head. In some implementations, an adjustable spray direction can be accomplished with, for example, a rotatable or otherwise adjustable nozzle to target the diffused vapor towards a user's face. In some cases, the positioning of the diffuser or the angling of the nozzle may consider a prevailing airflow, such as an upward draft from a shower fan so that the diffused vapor reaches the user's head.

Those of ordinary skill in the art will appreciate with reference to the present disclosure that other arrangements of the exterior of diffuser 100 are possible than those shown in FIGS. 1 to 6. For example, with reference to FIG. 6, other implementations of diffuser 100 may locate buttons 112 and 114 on an opposite side or may omit buttons 112 and 114 in place of another user interface. As another example variation, rear surface 118 may not be angled or slanted in other implementations.

FIG. 7 is a front view of diffuser 100 with the faceplate removed according to one or more embodiments. As shown in FIG. 7, the faceplate may be removably secured to a body of diffuser 100 via connectors 1501, 1502, 1503, and 1504 of base liquid tank 126, and via connectors 1521, 1522, and 1523 of the lower portion of the body of diffuser 100. The example of FIG. 7 also includes rocker bar 138 to facilitate easier removal of the faceplate by serving as a fulcrum for a user to push down on a bottom portion of the faceplate below rocker bar 138 to free the faceplate form connectors above rocker bar 138.

As discussed in more detail below, base liquid tank 126 is removable from the body of diffuser 100, such as for cleaning or filling. In the example of FIG. 7, base liquid tank 126 includes indentations 128a and 128b for a user to grip base liquid tank 126 and pull it from the body of diffuser 100. In some implementations, atomizer 102 may also be removed as part of base liquid tank 126. In addition, latch 148 may be depressed to release atomizer 102 from base liquid tank 126, such as for cleaning or for changing mesh component 1161.

As shown in the example of FIG. 7, atomizer 102 includes outer structure 147, nozzle 130, piezoelectric element 132, and mesh component 1161. During operation, circuitry 131 can control vibration of piezoelectric element 132, which at least partially surrounds mesh component 1161. In some implementations, circuitry 131 may vary a voltage supplied to piezoelectric element 132 to cause the piezoelectric element to vibrate at different frequencies, and as a result, cause mesh component 1161 to vibrate at different frequencies. The different vibration frequencies can provide different diffusion distances from atomizer 102 for an atomized liquid (e.g., a mixture of base liquid and oil).

Circuitry 131 is shown in dashed lines to indicate that it is contained within electronics enclosure 136 such that it is not visible when the faceplate of diffuser 100 is removed. In the example of FIG. 7, circuitry 131 includes one or more processors 133, at least one memory 135, and wireless interface 137 for wirelessly communicating with a remote device. Wireless interface 137 may provide, for example, Bluetooth connectivity and/or Wi-Fi network connectivity for communication with one or more remote devices, such as a smartphone or tablet. In addition, wireless interface 137 or other components of circuitry 131 may include NFC communication circuitry to wirelessly communicate with a remote device when in close proximity. Such wireless communication can facilitate, for example, launching an application on the remote device, initiating a preset aromatherapy routine, or initiating playback of audio (e.g., music or guided meditation) on the remote device.

As will be appreciated by those of ordinary skill in the art, circuitry 131 in some implementations can include a Printed Circuit Board (PCB) that can include, for example, one or more processors for executing instructions, such as a microcontroller, a Digital Signal Processor (DSP), an Application-Specific Integrated Circuit (ASIC), a Field Programmable Gate Array (FPGA), hard-wired logic, analog circuitry, and/or a combination thereof. In some implementations, circuitry 131 can include a System on a Chip (SoC), which may also include memory 135.

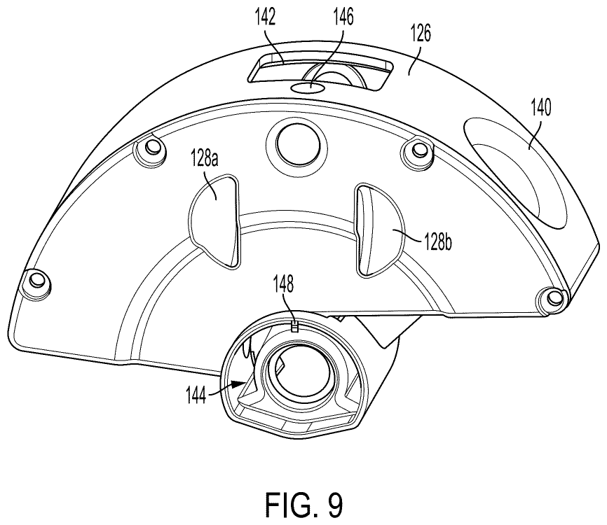

In addition, circuitry 131 can include a Real-Time Clock (RTC) in some implementations. The RTC of circuitry 131 can, for example, facilitate the timing of sessions indicating the duration of particular diffusion operating cycles and/or when sessions took place (e.g., time of day and on which day). This information may be communicated, along with other session information in some cases, to a remote device (e.g., a smartphone or tablet) via wireless interface 137 or other wireless communication circuitry (e.g., sensor 141 in FIG. 9) of diffuser 100 to provide the session information to an application executing on the remote device.

Such time information provided by an RTC can facilitate the tracking of usage patterns by time of day (e.g., morning vs. evening), which can enable, for example, personalized scent recommendations based on time-specific preferences (e.g., lavender at night, citrus in the morning), generating meaningful analytics (e.g., weekly morning routine patterns), syncing with health or wellness applications that analyze behavior chronologically, and supporting automatic cleaning reminders based on elapsed calendar time.

For its part, memory or memories 135 of circuitry 131 may include, for example, one or more Dynamic Random Access Memories (DRAMs) and/or various types of memory devices such as flash integrated circuits, NAND memory, NOR memory, Electrically Erasable Programmable Read-Only Memory (EEPROM), and/or other discrete Non-Volatile Memory (NVM) chips, or any combination thereof.

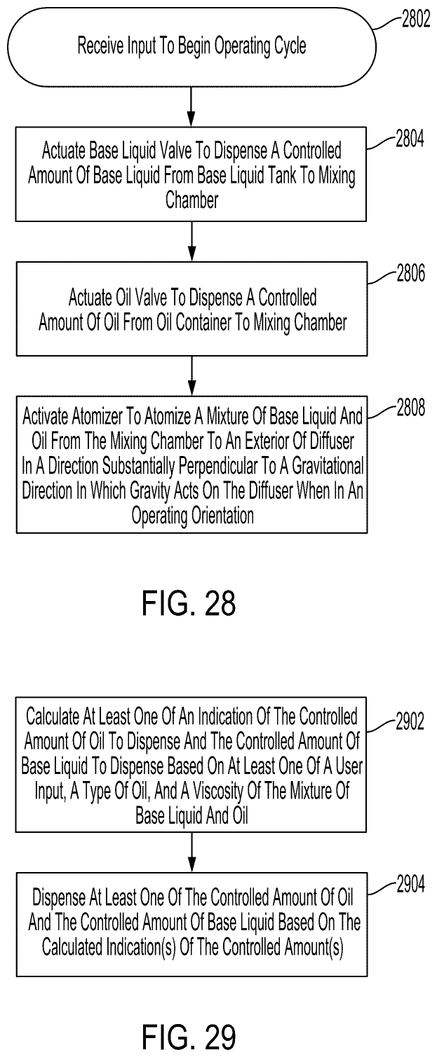

As discussed in more detail below with reference to processes of FIGS. 28 to 40, processor(s) 133 of circuitry 131 can execute firmware stored in memory or memories 135 to control operation of diffuser 100. Circuitry 131 is electrically connected to side buttons 112 and 114 to receive user input, such as to increase or decrease scent strength. Central button contact 151 also electrically connects to circuitry 131 and serves as a contact switch for receiving input from central button 104 shown in FIGS. 3 and 5.

Circuitry 131 is powered by one or more batteries located in battery compartment 134, which can provide additional protection against liquids and/or moisture. In some implementations, the one or more batteries of battery compartment 134 can be rechargeable, such as rechargeable lithium ion batteries. In such implementations, connection 120 can electrically connect to a charging device of battery compartment 134 to charge the battery or batteries of battery compartment 134. In other implementations, the one or more batteries located in battery compartment 134 may be wirelessly charged, such as by using inductive charging.

FIG. 8 is a close-up perspective view of an atomizer 102 according to one or more embodiments. As shown in FIG. 8, atomizer 102 includes outer structure 147 surrounding nozzle 130, which surrounds piezoelectric element 132, which in turn, surrounds mesh component 1161. In some implementations, latching notch 154 in outer structure 147 engages with latch 148 of FIG. 7 to hold atomizer 102 into contact with walls of a mixing chamber (e.g., walls of mixing chamber cavity 144 shown in FIG. 9) that holds a mixture of base liquid and oil during operation of diffuser 100. In this regard, an interior facing surface of mesh component 1161 forms part of the mixing chamber such that the mixture of base liquid and oil is in contact with the interior facing surface of mesh component 1161. As shown in FIG. 8, the rear walls of outer structure 147 may include a tapering and/or seals for press fitting atomizer 102 into mixing chamber cavity 144 to further secure atomizer 102 into mixing chamber cavity 144. In other implementations, latch 148 may be omitted and atomizer 102 may be sufficiently secured by press fitting atomizer 102 into place.

Vibration of piezoelectric element 132 causes atomization and propulsion of the mixture in the mixing chamber through holes in mesh component 1161 and out of nozzle 130. As noted above, circuitry 131 can control the vibration frequency of mesh component 1161, such as by varying a voltage supplied to piezoelectric element 132. For example, circuitry 131 may increase a voltage supplied to piezoelectric element 132 to increase a diffusion distance for an atomized vapor or mist from nozzle 130.

In some implementations, outer structure 147 includes a contact for circuitry 131 to supply a control signal (e.g., a voltage) for controlling the vibration of piezoelectric element 132. In other implementations, atomizer 102 can include a waterproof socket connector that can be disconnected for removing atomizer 102 from base liquid tank 126 and reconnected for reattaching atomizer 102 to base liquid tank 126.

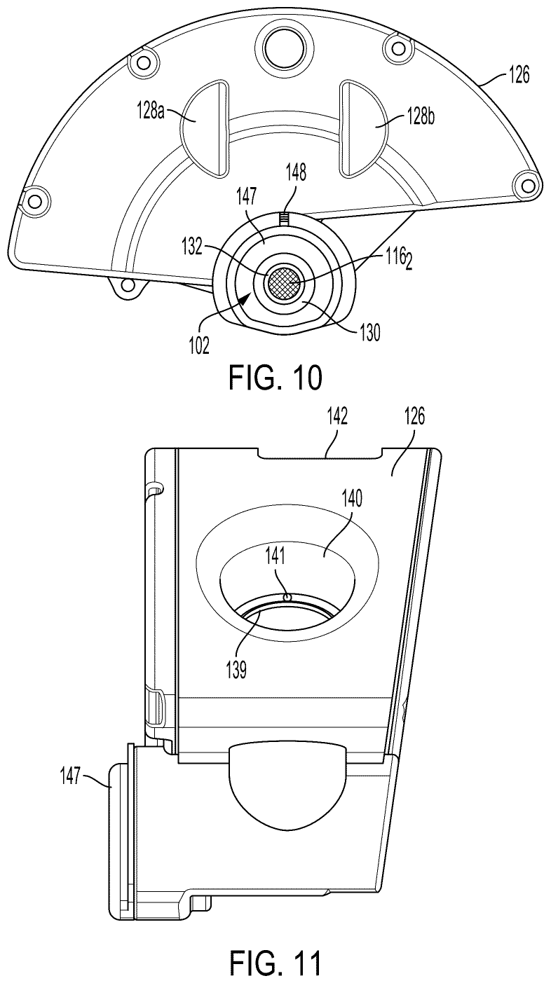

In addition, mesh component 1161 can be removed from nozzle 130 and outer structure 147 when atomizer 102 is removed from diffuser 100, as shown in FIG. 8. In some implementations, mesh component 1161 may be pushed by the user from the front through nozzle 130 to free mesh component 1161 from the rear. Mesh component 1161 may be repositioned from the rear of atomizer 102 or a different mesh component (e.g., mesh component 1162 shown in FIG. 10) may be repositioned in place of mesh component 1161.

FIG. 9 is a perspective view of base liquid tank 126 of diffuser 100 according to one or more embodiments. As shown in FIG. 9, base liquid tank 126 includes indentations 128a and 128b to facilitate insertion and removal of base liquid tank 126 to and from diffuser 100. Base liquid tank 126 also includes tank opening 142 for filling base liquid tank 126 with base liquid, and tank aperture 140 for inserting or removing an oil container. When base liquid tank 126 is inserted in diffuser 100, tank aperture 140 lines up with shell aperture 122 shown in FIG. 6, and tank opening 142 lines up with shell opening 124 shown in FIG. 6.

Mixing chamber cavity 144 provides a space for mixing base liquid dispensed from the main portion of base liquid tank 126 and oil dispensed from an oil container inserted into diffuser 100 via shell aperture 122 and base liquid tank aperture 140. As noted above, the full mixing chamber includes a front surface provided by the mesh component of atomizer 102 when latch 148 engages with latching notch 154 of atomizer 102 (shown in FIG. 8). In this regard, the front edges of mixing chamber cavity 144 can include a seal for sealing the rear surface of atomizer 102 onto mixing chamber cavity 144 and preventing leakage from the mixing chamber.

The volume of the mixing chamber, which is primarily defined by the volume of mixing chamber cavity 144, is significantly smaller than the volume of the main portion of the interior of base liquid tank 126 that holds the base liquid. In some implementations, the volume of base liquid tank 126 is greater than the volume of the mixing chamber by at least a factor of twenty. In some implementations, the volume of the mixing chamber can be within the range of four to eleven milliliters.

In addition to facilitating a generally horizontal (with respect to gravity) atomization of liquid, the relatively small volume of the mixing chamber (e.g., within the preferred range of four to eleven milliliters) can better conserve the amount of oil dispensed. In this regard, a smaller amount of oil can be added for a desired scent strength, as compared to a diffuser with a larger reservoir from which the liquid is vaporized. In addition, oil and base liquid can be periodically added into mixing chamber cavity 144 in a controlled manner throughout an operating cycle of the diffuser as needed so that the amount of oil dispensed is better tailored to the duration of the operation of diffuser 100, such as for five or eight minutes. This can result in the oil being added to the mixing chamber being fresher and providing a stronger scent for the atomized mixture, as opposed to the oil remaining in the mixture with the base liquid for a longer period of time and having a weaker scent for the atomized mixture.

The smaller volume of mixing chamber cavity 144 (e.g., within the preferred range of four to eleven milliliters) can also provide for a more concentrated amount of oil in the mixture as compared to conventional oil diffusers that use a larger reservoir. This can result in a stronger scent diffused from diffuser 100. In addition, the relatively small volume of mixing chamber cavity 144 (e.g., within the preferred range of four to eleven milliliters) can enable rinsing or flushing of the mixing chamber between uses with base liquid to keep the mixing chamber and the mesh component cleaner. In such rinse or flushing cycles, base liquid may be dispensed into mixing chamber cavity 144 without dispensing oil, and atomizer 102 may continue to diffuse mist or vapor from the mixing chamber for a period of time to clean the mixing chamber and the mesh component.

In the example of FIG. 9, base liquid tank 126 includes air vent opening 146 that fluidly connects at least one of the mixing chamber and an interior of the main portion of base liquid tank 126 to the exterior of diffuser 100. Air vent opening 146 relieves an air pressure in at least one of the mixing chamber and the main portion of the interior of base liquid tank 126. By relieving the air pressure, it is ordinarily possible to improve or increase the flow of base liquid from the interior of base liquid tank 126 into the mixing chamber. In this regard, an air vent facilitates the use of a relatively small mixing chamber, which may otherwise receive an insufficient flow of base liquid from base liquid tank 126 due to a build-up of pressure in the small mixing chamber.

The air vent can include a tube within base liquid tank 126 that runs from a top portion of mixing chamber cavity 144 through the interior of base liquid tank 126 and exits out of a top portion of base liquid tank 126 at air vent opening 146, as shown in FIG. 9 to fluidly connect the mixing chamber to the air outside of the diffuser. In some implementations, the air vent may further include a gap or perforations at a top portion the tube within base liquid tank 126 to fluidly connect the interior of the main portion of base liquid tank 126 above a base liquid level to the exterior air and to the top portion of mixing chamber cavity 144.

FIG. 10 is a front view of base liquid tank 126 including atomizer 102 inserted into base liquid tank 126 according to one or more embodiments. As shown in FIG. 10, atomizer 102 seals mixing chamber cavity 144 shown in FIG. 9. As discussed above, latch 148 of atomizer 102 can secure atomizer 102 into contact with the walls of mixing chamber cavity 144.

Atomizer 102 in the example of FIG. 10 includes a different mesh component 1162 than mesh component 1161 shown in FIG. 7. As noted above, the use of different mesh components can provide for different diffusion ranges or patterns by changing at least one of the quantity, size, and/or arrangement of holes. In some implementations, the mesh components can include electronically readable identifiers that allow circuitry of the diffuser or an application executed by a remote device to identify the mesh component used or to be used in the atomizer. In response to an input indicating use of the mesh component (e.g., a particular mesh pattern), the circuitry of the diffuser may adjust the operation of the diffuser to account for the different mesh patterns, such as by using different settings for oil concentration, a voltage supplied to the atomizer, and/or a rinse cycle duration or amount of base liquid used for a rinse cycle.

FIG. 11 is a side view of base liquid tank 126 according to one or more embodiments. As shown in FIG. 11, outer structure 147 of atomizer 102 protrudes from base liquid tank 126. In this regard, outer structure 147 protrudes from exterior shell 110 of diffuser 100 so that a surface of atomizer 102 forms part of an exterior surface of the diffuser when base liquid tank 126 is inserted into the diffuser. At least a portion of the surface of the atomizer or mesh component diffuses the atomized liquid from the mixing chamber directly to air that is external to the diffuser. This allows for the direct diffusion of liquid (e.g., a mixture of base liquid and oil or base liquid without oil) directly from the mixing chamber via mesh component 1162 to an exterior of the diffuser. In contrast to medical inhalers or nebulizers that may use a mask or nasal interface (e.g., a nasal cannula) to diffuse an atomized liquid without diffusion directly to the surrounding air, diffuser 100 does not require physical contact with the user, such as through a mask or nasal interface that can interfere with the user's enjoyment of an immersive aromatic experience. In this regard, the arrangement of the mixing chamber and atomizer 102 can diffuse a scented mist or vapor directly to the surrounding air, such as to a configurable diffusion distance of twenty to one hundred centimeters from the diffuser.

In the example of FIG. 11, base liquid tank aperture 140 includes sensor 141. In some implementations, sensor 141 can include an optical sensor or a radio sensor. As a radio sensor, sensor 141 can include, for example, an NFC sensor or an RFID sensor configured to receive information from a tag on an oil container that can provide information via sensor 141 to circuitry 131 of the diffuser about the oil container inserted into tank aperture 140. Such information can include, for example, a type of oil, a manufacturer of the oil, an expiration date or manufacturing date of the oil, and/or a volume of oil supplied in the oil container.

In some implementations, sensor 141 as an NFC sensor may operate as an NFC transceiver capable of bidirectional communication. In addition to reading NFC tags, such as from an oil container or oil receptacle, sensor 141 may communicate or otherwise interact with a nearby remote device (e.g., a smartphone). The interaction with sensor 141 may cause, for example, the launching of an application on the remote device, the provision of usage recommendations, initiating audio playback through the remote device, or data transmitted to the remote device, such as session information (e.g., a time or duration of a session).

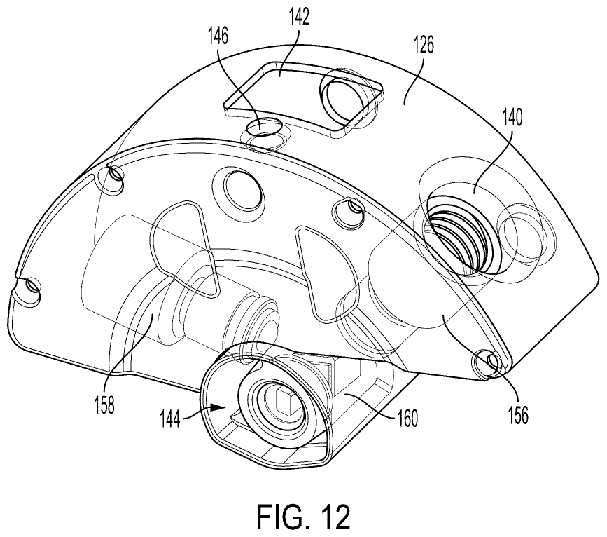

As an optical sensor, sensor 141 may be used to read a barcode or QR code from an oil container to obtain information, such as the information provided above, for circuitry 131 about the oil container inserted into tank aperture 140. The circumferential placement of sensor 141 may correspond to the location of the barcode or QR code on the oil container after being screwed into a receptacle within base liquid tank 126 (e.g., receptacle 156 in FIG. 12). In other implementations, sensor 141 may be omitted. In such implementations, sensor 141 may be omitted in favor of obtaining information about the oil container from another source, such as an application executing on a remote device that communicates with diffuser 100 and is capable of retrieving information from the oil container, such as through a camera or NFC receiver of the remote device.

FIG. 11 also depicts contact sensor 139, which may facilitate the use of an oil container as a joystick for providing user input. In operation, contact sensor 139 receives a mechanical input from movement of the oil container and provides one or more electrical signals to circuitry 131 indicating movement of the oil container in one or more directions. Circuitry 131 may then control operation of diffuser 100 based on the electrical signal(s).

In some implementations, contact sensor 139 may include a gimbal assembly with potentiometers for detecting movement of the oil container and providing an electrical signal to circuitry 131. Movement of the oil container by the user can then provide instructions, such as to increase or decrease a scent intensity or a duration of a current operation cycle. In some implementations, contact sensor 139 may also be used by circuitry 131 to detect insertion of the oil container. Pressing down on the oil container may also provide a confirming selection input in some implementations. In other implementations, contact sensor 139 may be omitted such that user input is provided by other buttons, such as central button 104 and/or buttons 112 and 114.

FIG. 12 is a translucent top perspective view of base liquid tank 126 including base liquid connector 158 and receptacle 156 for receiving an oil container according to one or more embodiments. As shown in FIG. 12, each of base liquid connector 158 and receptacle 156 connects to the mixing chamber to dispense base liquid and oil, respectively, into the mixing chamber. In the example of FIG. 12, an interior of receptacle 156 includes threading for screwing an oil container into the receptacle. In other implementations, a different attachment configuration can be used for the oil container, such as a catch of receptacle 156 that engages a ridge on the oil container to secure the oil container into receptacle 156.

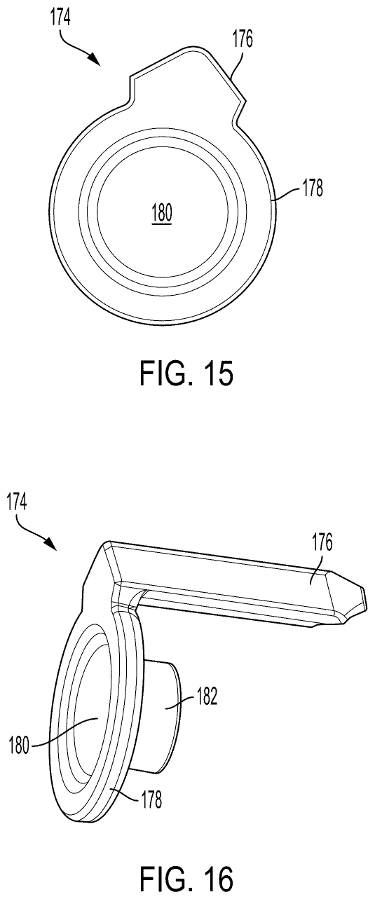

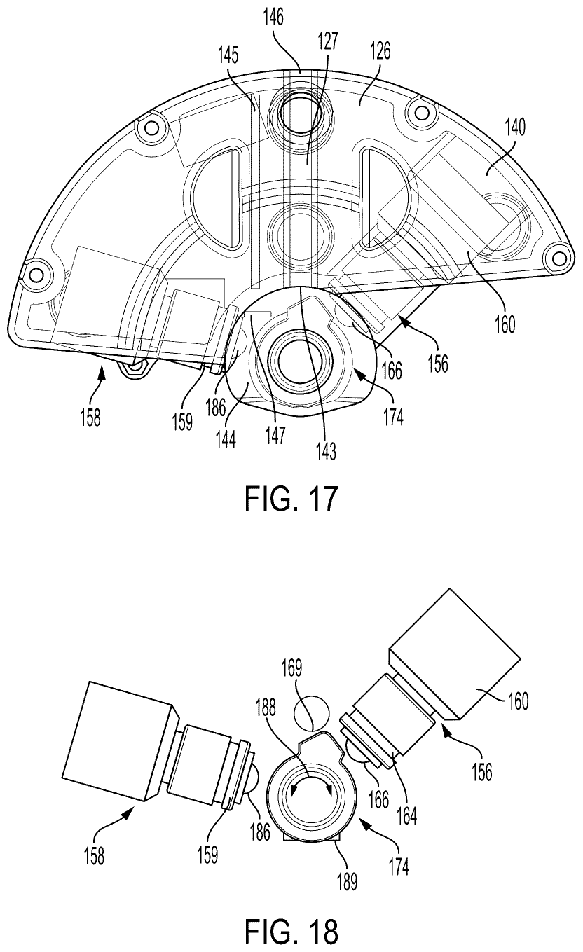

As discussed in more detail below, motor 190 controls an actuator (e.g., actuator 174 in FIGS. 15 and 16) that contacts a base liquid valve (e.g., base liquid valve 159 in FIG. 17) at an end portion of base liquid connector 158 closest to the mixing chamber to control the amount of base liquid dispensed into the mixing chamber. In addition, the actuator can be actuated to contact an oil valve (e.g., oil valve 164 in FIG. 17) to control the amount of oil dispensed into the mixing chamber. In the example of FIG. 12, the flow of the oil and the flow of the base liquid can result from opening of the base liquid valve and opening of oil valve to allow gravity to induce flow of the base liquid or the oil. In some implementations, the angles or tilt of receptacle 156 and base liquid connector 158 with respect to the gravitational direction can be based at least in part on desired flow rates of the base liquid and the oil. In this regard, the longitudinal centerline of receptacle 156 is more vertical or more aligned with the gravitational direction than the longitudinal centerline of base liquid connector 158 due to the greater viscosity of the oil than the base liquid, which flows more freely than the oil. For example, a channel within base liquid connector 158 may be angled for a particular drainage rate of base liquid from the main portion of the base liquid tank.

While the angle of the channel within receptacle 156 or base liquid connector 158 facilitates flow, the actual flow rate may not be determined solely by gravity. In addition to the angle of the channel, the flow of base liquid or oil can also be controlled by, for example, a combination of the base liquid valve or oil valve opening size and the duration of actuation (i.e., the time the actuator presses onto the valve), and/or fluid pressures inside the mixing chamber, base liquid tank, and/or oil container.

The foregoing arrangement of the mixing chamber, base liquid tank 126, receptacle 156, and base liquid connector 158 facilitates the use of gravity so as to provide a consistent flow of liquid without requiring active pumping mechanisms, which reduces the complexity, cost, and power consumption of the diffuser. This arrangement may also simplify cleaning and maintenance by not requiring the flushing of internal pumps, pipes, or wicks that may otherwise be used in pump-based systems. However, other implementations may include additional actuators or motors that control fluid flow from separate sources that may include pump-based systems.

As will be appreciated by those of ordinary skill in the art with reference to the present disclosure, different arrangements of base liquid tank 126 are possible than those shown in FIGS. 9 to 12. For example, other implementations of base liquid tank 126 may include, for example, a cylindrical shape for base liquid tank 126 as opposed to a more semi-circular shape or may include two motors for separately actuating the base liquid valve and the oil valve. In this regard, the base liquid tank is not limited to a specific tank shape. For example, if a non-gravity-fed system is implemented—such as one using a pump or pressurized delivery—the base liquid tank may be positioned in a different orientation, and the shape or structure of the base liquid tank may vary significantly.

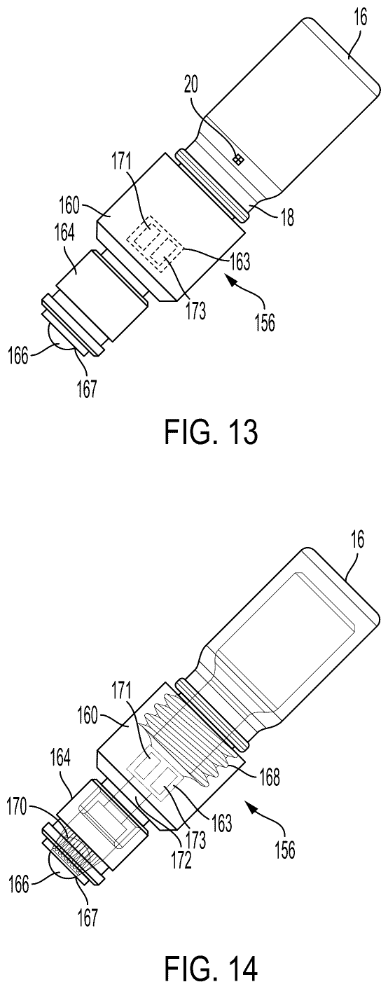

FIG. 13 is a close-up view of receptacle 156 and its oil valve 164 according to one or more embodiments. As shown in FIG. 13, oil container 16 is inserted into receptacle 156 by screwing oil container 16 into collar 160 of receptacle 156. In the example of FIG. 13, oil container 16 includes barcode or QR code 20 and tag 18 for providing information about the oil container to circuitry 131 of diffuser 100 via sensor 141.

Oil valve 164 of receptacle 156 includes ball 166 that is spring-actuated by an actuator contacting ball 166 to create space 167 between an opening of valve 164 and ball 166 when it is pushed into the valve by the actuator. As discussed in more detail below with reference to FIGS. 17 and 18, the actuator may contact ball 166 for a predetermined time to allow a controlled amount of oil to flow from oil container 16 through space 167. When the actuator moves away from contacting ball 166, space 167 is closed to stop the flow of oil by ball 166 moving to its default position.

In some implementations, receptacle 156 is removable from base liquid tank 126. In such implementations, a user may screw or otherwise attach receptacle 156 onto oil container 16 after removing a cap from the oil container. This can allow for the oil container to remain upright while the cap is removed to prevent spilling of the oil. Receptacle 156 then provides a seal to keep the oil from escaping oil container 16 as the combination of receptacle 156 and oil container 16 is inverted and inserted into shell aperture 122 and tank aperture 140 to engage with the mixing chamber inside base liquid tank 126. In some implementations, receptacle 156 may engage and be secured to the mixing chamber using, for example, a reverse threading into the mixing chamber, a bayonet lock, or a clip.

In some implementations, receptacle 156 includes receptacle tag 163, which includes memory 171 and communication circuitry 173. In such implementations, one or more user settings or preferences associated with a user can be stored in memory 171. The user settings may then be transmitted by communication circuitry 173 to circuitry 131, such as via sensor 141. In this regard, receptacle tag 163 can include, for example, an NFC tag or an RFID tag to provide circuitry 131 of diffuser 100 with user settings and/or preferences of a particular user of diffuser 100.

For example, receptacle tag 163 can store preferences or user settings, such as a personalized default operating cycle time (e.g., eight minutes), a personalized scent intensity indication (e.g., level 3 out of 5) corresponding to the controlled amount of oil dispensed from the oil container during use, personalized lighting settings for lighting ring 106 (e.g., breathing guidance lighting enabled), and/or a preferred diffusion distance or range (e.g., 40 centimeters).

In some implementations, the user may set their settings or preferences with an application executing on a remote device, such as a smartphone or tablet. The remote device may wirelessly communicate these user settings to circuitry 131 via wireless interface 137 shown in FIG. 7. The user settings may be stored in memory 171 of receptacle tag 163 from circuitry 131 of diffuser 100 via sensor 141 or wireless interface 137 of circuitry 131. In other cases, the remote device may wirelessly transmit the user settings directly to receptacle tag 163 via communication circuitry 173 without involving circuitry 131 of diffuser 100. In this regard, the communication between receptacle tag 163 and circuitry 131 or a remote device may be bidirectional, such as where NFC is used for communication circuitry 173 of receptacle tag 163. In some implementations, the importation of user settings by circuitry 131 from receptacle tags can be enabled or disabled, such as via the remote device or through central button 104, and/or side buttons 112 and 114.

In implementations where receptacle 156 includes receptacle tag 163, a different user of diffuser 100 may have a different receptacle that stores their preferences or user settings that they can use to attach to oil containers. In addition, the receptacles may include a visual indicator, such as different colors for their collars to visually indicate the users associated with the receptacle and distinguish their receptacle from another receptacle. Circuitry 131 of diffuser 100 can distinguish between the different users based on reading information from the different receptacle tags, such as by using sensor 141 or wireless interface 137. The different receptacles can then be used to set different user settings for operating diffuser 100 based on the user's preferences or settings.

FIG. 14 is a transparent close-up view of receptacle 156 illustrating internal components of the receptacle and its oil valve according to one or more embodiments. As shown in FIG. 14, collar 160 includes internal threading 168 for securing oil container 16, and receptacle tag 163 for communicating with circuitry 131 of diffuser 100. The example of FIG. 14 also shows the internal space of oil container 16. In some implementations, the volume of oil contained in oil container 16 can include 10 milliliters (ml) or 15 ml.

As shown in the example of FIG. 14, collar 160 of receptacle 156 includes internal channel 172 for supplying oil from container 16 to oil valve 164. Ball 166 of oil valve 164 is biased by spring 170 towards opening 167 of receptacle 156 such that ball 166 seals or otherwise blocks the oil in channel 172 from escaping receptacle 156. The stiffness or spring constant of spring 170 may be chosen based on a resistance supplied by an actuator of diffuser 100 (e.g., actuator 15 in FIG. 15) for releasing oil from oil valve 164. In operation, the actuator may contact and depress ball 166 at least partially into receptacle 156 to enable oil to flow from or be released from opening 167.

Those of ordinary skill in the art will appreciate with reference to the present disclosure that other arrangements of receptacle 156 are possible. For example, other implementations of receptacle 156 may not include receptacle tag 163, may use a different method of securing oil container 16, and/or may use a different type of valve for oil valve 164.

FIG. 15 is a front view of actuator 174 according to one or more embodiments. As shown in FIG. 15, actuator 174 includes contact surface 176, front face 180, and outer ring 178. Contact surface 176 provides a surface to contact oil valve 164 and the base liquid valve (e.g., base liquid valve 159 shown in FIG. 17) of base liquid connector 158. In the example of FIG. 15, contact surface 176 includes two halves configured to apply an increasing amount of pressure onto a ball of each of the base liquid valve and oil valve 164. In some implementations, the ball may initially be depressed by contacting an edge of contact surface 176 and further depressed with a greater pressure as the actuator moves into contact with the ball near the apex of contact surface 176. The greater pressure on the ball increases the size of the opening of the valve to allow more liquid to be dispensed.

It should be understood that the shape or profile of contact surface 176 is not limited to the illustrated example. Variations in the contour or geometry of contact surface 176 may be used to adjust the rate of flow of oil or base liquid when the actuator engages the corresponding valve. Different shapes may produce different pressure profiles or contact durations, thereby affecting the amount or speed of liquid dispensed.

Front face 180 in the example of FIG. 15 is indented to provide additional space behind atomizer 102 when inserted into the center of mixing chamber cavity 144. In this regard, the mixture of base liquid and oil behind atomizer 102 is vaporized or atomized through mesh component 116 to be diffused from nozzle 130 of atomizer 102. In other implementations, front face 180 may differ and still create or preserve adequate space behind the atomizer for vaporization of the liquid mixture. The shape of front face 180 may therefore be planar, recessed, angled, or otherwise contoured.

As discussed in more detail below, actuator 174 is arranged within diffuser 100 to dispense both a controlled amount of oil and a controlled amount of base liquid using a single actuator and motor assembly. In other implementations, diffuser 100 may use a separate actuator for each of the oil valve and the base liquid valve, or more than two actuators may be employed to independently control additional valves from additional liquid sources (e.g., additional oil containers). This configuration enables more complex fluid control scenarios, such as real-time mixing of different essential oils or separate control of functional additives and oils. Each actuator can be calibrated for specific timing, volume, and ratio requirements, allowing for dynamic scent blending and highly personalized diffusion routines.

FIG. 16 is a perspective view of actuator 174 according to one or more embodiments. As shown in FIG. 16, actuator 174 further includes seal 182 that is configured to provide a liquid seal to keep liquid in the mixing chamber. In operation, actuator 174 rotates between at least a first position and a second position with the first position being a contact point with the base liquid valve and the second position being a contact point with oil valve 164. In some implementations, additional positions may be defined within the range of the rotation of actuator 174, such as a home position between the first and second positions or an air vent position that relieves a pressure in the mixing chamber.

FIG. 17 is a transparent front view of base liquid tank 126 illustrating internal components of the base liquid tank including actuator 174 that contacts oil valve 164 and base liquid valve 159 according to one or more embodiments. As shown in FIG. 17, actuator 174 is inserted into a center of mixing chamber cavity 144. In operation, a motor rotates actuator 174 to contact ball 166 of oil valve 164 for a period of time corresponding to a target scent strength and rotates to contact ball 186 of base liquid valve 159 for a period of time corresponding to a base liquid volume that may be based, for example, on a volume of the mixing chamber, a duration for an operating cycle, a target concentration or ratio of oil to base liquid, and/or a viscosity for the mixture of oil and base liquid.

In some implementations, actuator 174 is removable from base liquid tank 126, as is atomizer 102 for cleaning. In this regard, actuator 174 may include a hollow space opposite front face 180 that is centered about a center line of seal 182 and/or outer ring 178 for removably attaching actuator 174 onto a motor shaft for rotating actuator 174.

Base liquid tank 126 in FIG. 17 further includes base liquid level sensor 145 and mixing chamber liquid level sensor 149 to detect liquid levels in each of an interior of the main portion of base liquid tank 126 and mixing chamber cavity 144, respectively. In some implementations, base liquid level sensor 145 and mixing chamber liquid level sensor 149 can include, for example, floating contacts that provide a relative resistance or otherwise modify an electrical signal to indicate a level of the liquid. In other implementations, base liquid level sensor 145 and mixing chamber liquid level sensor 149 may use light reflection or other means of detecting a liquid level.

Air vent 127 in FIG. 17 provides a pressure relief to facilitate the dispensing of base liquid and oil into mixing chamber cavity 144. In the example of FIG. 17, air vent mixing chamber opening 143 in a top portion of mixing chamber cavity 144 is fluidly connected via air vent 127 to air vent opening 146 that is open to an exterior air pressure outside of diffuser 100. Air vent 127 can include, for example, a cylindrical tube that crosses through an interior of the main portion of base liquid tank 126.

FIG. 18 illustrates the actuation of actuator 174 between contacting ball 166 of oil valve 164 and contacting ball 186 of base liquid valve 159 according to one or more embodiments. As shown in FIG. 18, actuator 174 rotates in radial directions 188 and can move back and forth between a position where the apex of contact surface 176 of actuator 174 contacts ball 166 of oil valve 164 and contacts ball 186 of base liquid valve 159. In other implementations, actuator 174 may be arranged to rotate in a horizontal plane instead of a vertical plane as depicted in FIG. 18.

In the example of FIG. 18, the apex of contact surface 176 may also move to position 169, which may serve as a home or default position when not dispensing base liquid or oil into mixing chamber cavity 144. In other examples, position 169 may serve as a rinse position for more quickly dispensing base liquid from the main portion of base liquid tank 126 than the more controlled dispensing of base liquid from base liquid valve 159. In yet another example, position 169 can provide either a seal or an opening to air vent 127 to control an air pressure in the mixing chamber. In some implementations, the actuation of actuator 174 can move to multiple positions such as a vent position, a home position, and/or a rinse position, in addition to an oil dispensing position and a base liquid dispensing position.

In other implementations, and as noted above, multiple actuators may be used within the diffuser to independently control multiple oil valves connected to different oil containers. This configuration can enable real-time mixing of multiple essential oils to generate customized blends based on user preferences or dynamic recipes stored in memory (e.g., memory 135 of circuitry 131). Each actuator can be controlled with specific timing and dosage parameters to achieve precise multi-oil diffusion, enabling advanced scent layering and aromatherapy protocols.

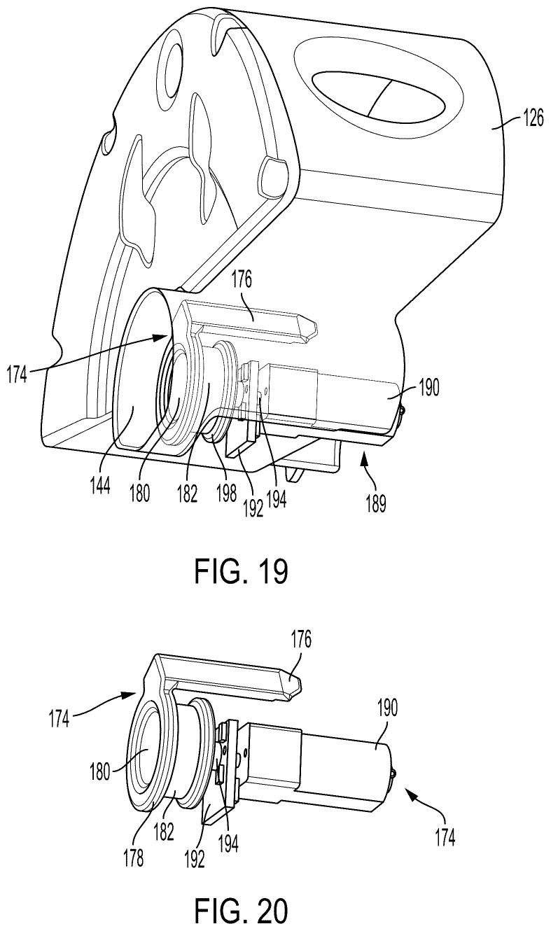

FIG. 19 is a transparent bottom perspective view of base liquid tank 126 illustrating an internal arrangement of actuator 174 and motor assembly 189 according to one or more embodiments. FIG. 20 is a perspective view of the internal arrangement of actuator 174 and motor assembly 189 shown in isolation from base liquid tank 126.

As shown in FIGS. 19 and 20, motor assembly 189 includes motor 190, motor shaft 194, and position sensor 192. Actuator 174 is attached to motor shaft 194 for rotation by motor assembly 189, which may be powered by a battery of diffuser 100 and controlled by circuitry 131 of diffuser 100. Position sensor 192 can provide feedback on the position of motor shaft 194 to control rotation of motor shaft 194 via motor 190 of motor assembly 189. In some implementations, position sensor 192 can include a Hall sensor.

In some implementations, the rotation of motor shaft 194 can be controlled to provide different degrees of pressure onto ball 166 of oil valve 164 and onto ball 186 of base liquid valve 159. The different amounts of pressure can provide a more controlled dispensing of liquid from the valves in addition to the timing for releasing the liquid from the valve, such as an overall contact time of two seconds with the oil valve and four seconds with the base liquid valve to provide a calculated or estimated volume for both liquids with a particular concentration of oil. As noted above, the relatively small size of mixing chamber cavity 144 can conserve the usage of oil and better enables atomizer 102 to diffuse a vapor or mist in a generally horizontal direction toward the user, in addition to facilitating a more effective rinsing of mixing chamber cavity 144 between uses, which can prevent cross contamination of oils.

In some implementations, actuator control and motor operation may be commanded by a remote device, such as a smartphone or tablet, executing an application. The application may wirelessly communicate with circuitry 131 via wireless interface 137 to adjust actuator positions, initiate preset diffusion programs, or transmit user-specific blending preferences. This functionality enables a more personalized and remote-controlled user experience and can enable firmware logic to respond dynamically to real-time input from the remote device.

The present disclosure can include mobile device interfaces as part of the diffusion system and should not be limited to the diffuser's onboard physical buttons alone. As discussed above, the diffusion system may support Bluetooth, Wi-Fi, or NFC-based command pathways.

### Example Network Environment

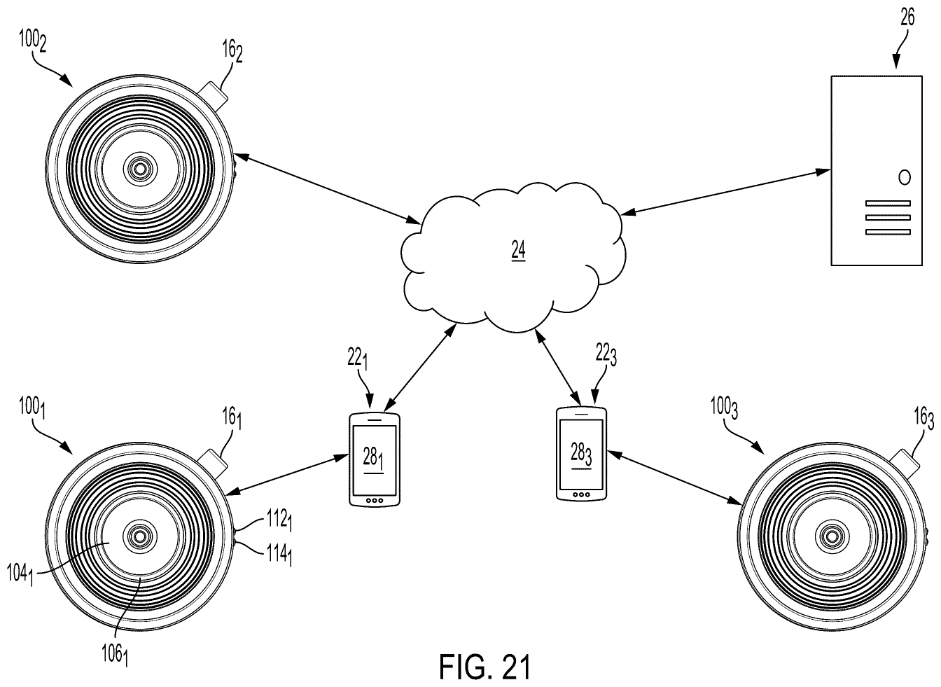

FIG. 21 illustrates an example network environment including diffusers 100, remote devices 22, and server 26 according to one or more embodiments. As shown in FIG. 21, diffuser 1002, and remote devices 221 and 223 communicate via network 24, which can include, for example, a Local Area Network (LAN) or a Wide Area Network (WAN), such as the internet. Each of remote devices 221 and 223 includes a user interface 281 and 283, respectively, that serve to both display information to a user and receive user input from the user, such as where user interfaces 28 are touchscreens. In the example of FIG. 21, remote devices 22 are shown as smartphones, but one or both of remote devices 22 can include, for example, a tablet or other type of computing device, such as a laptop, desktop, or wearable device, such as a Virtual Reality (VR) headset or a smartwatch.

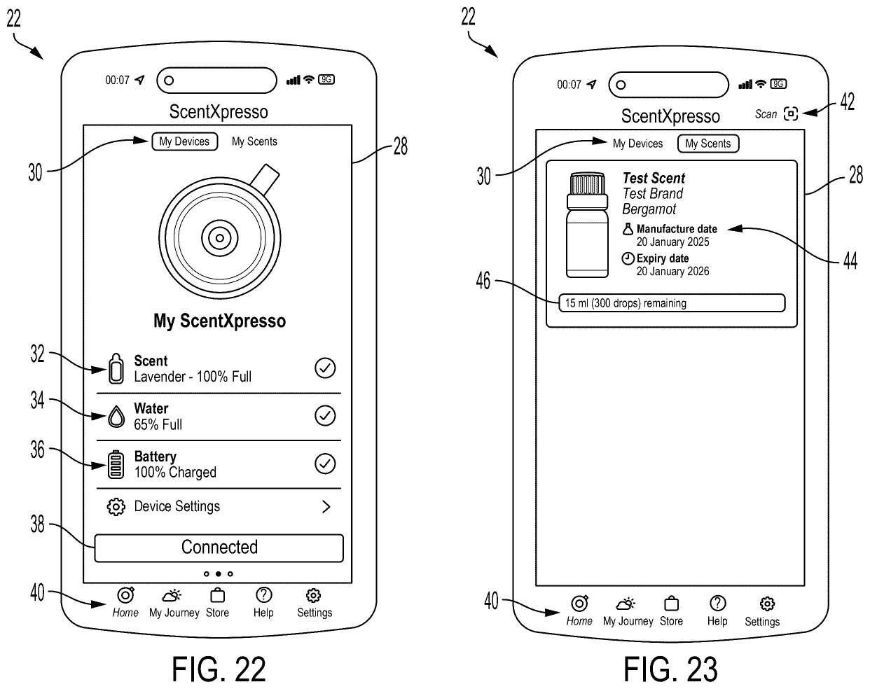

As discussed in more detail below with reference to FIGS. 22 to 27, each of remote devices 221 and 223 can execute an application that enables communication with each of diffusers 1001 and 1003, respectively, and configuration or control of the diffuser. Each remote device 22 can wirelessly communicate with its respective diffuser 100 via wireless interface 137 of circuitry 131 of the diffuser, and/or communicate via a cable connected to connection 120 of the diffuser. In this regard, each pair of remote device 22 and diffuser 100 can be geographically located within proximity of each other when the remote device communicates with the diffuser. However, the use of the application executed by the remote device may not require communication with the diffuser and may provide off-line configuration of the diffuser's operation.

In some implementations, the remote device application may communicate not only with the diffuser directly, but also with a cloud-based service or platform (e.g., provided by server 26 in the example of FIG. 21). In such implementations, server 26 may manage synchronized user profiles, enable cross-device control, and support remote scheduling or environmental automation based on user location and/or preferences. For example, a user's diffusion settings may be saved from a remote device 22 for storage in server 26 and may be applied when the user logs into the platform from a different remote device, or when a different diffuser is used in another location.

Additionally, the platform may support Firmware-Over-The-Air (FOTA) updates via server 26, allowing new features, bug fixes, and/or scent blending settings to be deployed without requiring physical access to the diffuser. These capabilities provide a scalable architecture for managing multiple diffusers across households or commercial environments.

In the example of FIG. 21, diffuser 1002 communicates directly with network 24, such as through a Wi-Fi connection provided by wireless interface 137 of diffuser 1002. In this regard, each diffuser 100 in the example of FIG. 21 may be capable of communicating directly with network 24 and indirectly with network 24 via a remote device 22.

As shown in FIG. 21, diffuser 1001 includes central button 1041, buttons 1121 and 1141 for receiving user input at diffuser 1001. Diffuser 1001 in some implementations may communicate user input it receives back to remote device 221 to provide two-way communication between diffuser 1001 and remote device 221. In addition, diffuser 1001 includes lighting ring 1061 to provide feedback and/or other information, such as breathing guidance or synchronization with audio played or otherwise initiated from remote device 221 (e.g., music, guided meditation, or ambient sounds such as thunder, waves, or birdsong).

Diffusers 1002 and 1003 in the example of FIG. 21 provide similar functions as diffuser 1001. In the example of FIG. 21, each of diffusers 1001, 1002, and 1003 have different oil containers 161, 162, and 163, respectively, inserted into an oil receptacle of the diffuser, which can contain different types of essential oils to provide different scents for diffusion.

Server 26 communicates via network 24 to obtain information provided by remote devices 22 and diffuser 1002, such as user preferences, settings, purchases of oils or accessories, and/or usage history of diffusers 100 by different users. In addition, server 26 may communicate with remote devices 22 and diffuser 1002 via network 24 to provide updates to applications executed by remote devices and/or to a firmware of diffusers 100.

As discussed in more detail with reference to the recommendation process of FIG. 39, server 26 may collect information on oil usage history from multiple users of diffusers 100 and send recommendations to applications executed by remote devices 22 for oils directed to particular users to try based on their usage history and/or other information provided by the user, such as a desired goal (e.g., relaxation, increasing alertness) and/or user-supplied ratings on one or more oils used by the user in the diffuser 100. In some implementations, server 26 may use machine learning to provide recommendations with an Artificial Intelligence (AI) model that may be trained (e.g., with fine-tuning) using information collected from remote devices and/or diffusers for many users.

Server 26 may also coordinate AI-enhanced personalization across devices, enabling a user's diffuser preferences to evolve based on cumulative feedback and usage patterns across time and locations. For example, if a user consistently rates citrus oils highly during morning sessions, the application or platform may prioritize similar oils during future morning routines. Data from multiple users can be anonymized and used for collaborative learning to improve the recommendation engine across the platform.

Those of ordinary skill in the art will appreciate with reference to the present disclosure that the network environment of FIG. 21 is for the purposes of illustration and that, in practice, many more remote devices and diffusers may communicate with each other and with network 24. In some implementations, a diffuser, such as diffuser 1001, may communicate with multiple remote devices 22 for the same user or for different users of diffuser 1001.

### Example Graphical User Interfaces

FIG. 22 illustrates an example device status graphical user interface displayed on user interface 28 of remote device 22 according to one or more embodiments. As shown in FIG. 22, top menu bar 30 facilitates selection between providing information about a diffuser (e.g., diffuser 100) in communication with remote device 22 (i.e., “My Devices”) and providing information on one or more oils used in the diffuser and/or another diffuser, or otherwise associated with the user (i.e., “My Scents”). As discussed above, remote device 22 may communicate wirelessly (e.g., via Bluetooth or Wi-Fi) with the diffuser or may communicate via a wired connection (e.g., via USB).

The device status under “My Devices” provides oil information 32 indicating a scent and amount of oil remaining in an oil container inserted into the diffuser, base liquid level information 34 indicating an amount of base liquid remaining in a base liquid tank of the diffuser, and battery charge information 36 indicating a charge level of a battery of the diffuser. In addition, the device status includes communication connection status 38 to indicate whether remote device 22 is communicatively connected to the diffuser. Communication connection status 38 may change to “Disconnected” when a communication link is lost between remote device 22 and the diffuser, such as when the diffuser is outside of a wireless communication range, or the remote device has been put into to an airplane mode.

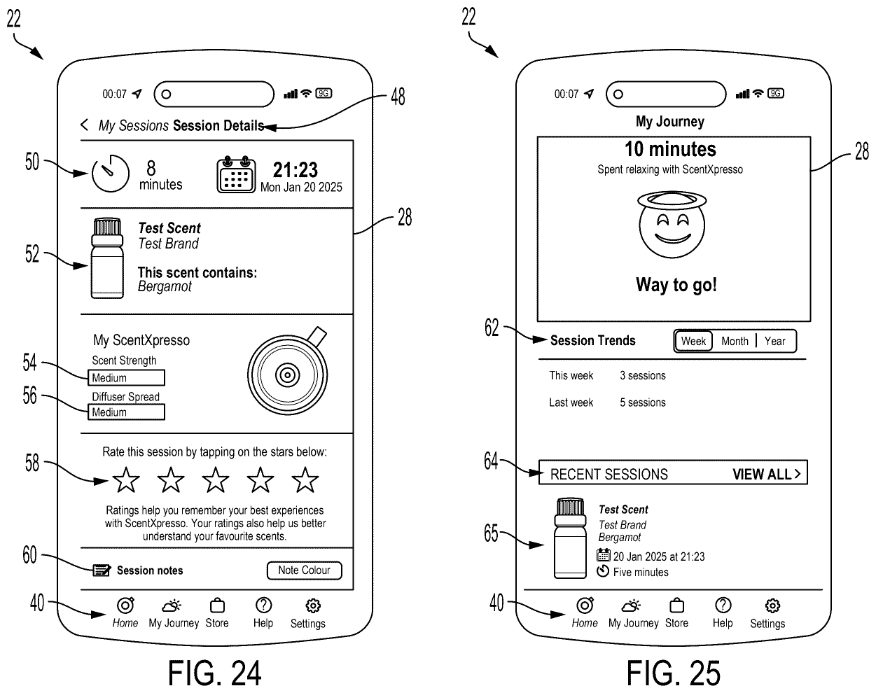

In the example of FIG. 22, bottom menu bar 40 facilitates selection between different graphical user interfaces for display on user interface 28. As shown in FIG. 22, bottom menu bar 40 can allow a user to switch between a home screen, which may be the device status shown in FIG. 22, a personalized session history screen for the user (i.e., “My Journey” in FIG. 22), a store screen for ordering accessories for the diffuser and/or different oils to use with the diffuser, a help screen for providing information or troubleshooting for using the diffuser or for the application being executed on remote device 22, and one or more setting screens, such as those shown in FIGS. 24 and 25 discussed below.

FIG. 23 illustrates an example scent information graphical user interface displayed on user interface 28 of remote device 22 according to one or more embodiments. As shown in FIG. 23, the selection for top menu bar 30 has been changed from “My Devices” to “My Scents” and user interface 28 displays information about an oil container associated with the user.

In some cases, the oil container may have been purchased by the user via a storefront accessed through the application providing the graphical user interface, and the information about the oil container is provided for display on user interface 28. In other cases, the oil container may be recognized by the diffuser, such as with sensor 141 discussed above with reference to FIG. 11. The diffuser may, for example, optically read data or otherwise wirelessly receive data from an oil container when the oil container is inserted into the diffuser. In yet other cases, a user may initiate the collection of information about the oil container by selecting scan button 42 and optically reading or otherwise wirelessly reading data from an oil container, such as from a QR code captured by a camera of remote device 22. The various methods of obtaining information about the oil container may overlap, such as where information is provided on an oil container purchased by the user, and the information may be updated or augmented by data received via the diffuser when the oil container is used in the diffuser.

The scent information in FIG. 23 includes oil container details 44, which includes a brand for the oil, a scent for the oil (e.g., “Bergamot” in FIG. 22), a manufacture date for the oil, and an expiry date for the oil. In addition, the scent information includes remaining amount indicator 46. As discussed in more detail below with reference to the remaining liquid determination process of FIG. 34, the diffuser may track the amount of oil dispensed from the oil container and calculate or estimate a remaining amount of oil in the oil container. This estimate or calculation may be provided from the diffuser to remote device 22. In other implementations, the diffuser may only provide remote device 22 with a dispensed amount of oil, and the application executing on remote device 22 may calculate the remaining amount.

In implementations where the diffuser can uniquely identify different oil containers, the application executing on remote device 22 can update remaining amount indicator 46 despite intervening uses of other oil containers in the diffuser. In such implementations, the usage of an oil container can be tracked among multiple diffusers such that remaining amount indicator 46 can indicate usage from more than one diffuser. In other implementations, remaining amount indicator 46 may simply provide an indication of the oil container volume, such as 10 ml or 15 ml without calculating or estimating a remaining amount of oil.

FIG. 24 illustrates an example session details graphical user interface displayed on user interface 28 of remote device 22 according to one or more embodiments. The session details can include information about a current operating cycle of the diffuser and/or a selected session history from previous operating cycles of the diffuser. In some implementations, the session details may be accessed by selecting a button 48 on a top menu, for example.

As shown in the example of FIG. 24, the session details can include duration control 50, scent information 52, scent strength control 54, diffuser spread control 56, session rating 58, and session notes 60. Duration control 50 can include a time setting for an upcoming session that may be configurable for the length of time the session or operating cycle will last or may indicate a remaining time for a current session in progress. As discussed above, a user may set a session time and may then increase or decrease the time of a current session in progress using buttons on the diffuser in some implementations. In addition, the duration control 50 may also include the date and time when the session occurred, as shown in FIG. 24. Scent information 52 can provide the name of the brand of the oil and the scent of the oil used for the session.

Scent strength control 54 can provide a way for the user to increase or decrease the controlled amount of oil added to the mixture of base liquid and oil to vary the strength of the scent diffused from the diffuser. In some implementations, scent strength control 54 can include a drop down menu or a slider to increase or decrease the scent strength for an upcoming or current session/operating cycle of the diffuser.

Diffuser spread control 56 can provide a way for the user to increase or decrease the diffusion distance or range of the mist or vapor diffused from the diffuser. In some implementations, scent strength control 54 can include, for example, a drop down menu or a slider to increase or decrease the diffusion distance. As discussed above, the diffusion distance may be controlled by a frequency of vibration of a piezoelectric element of an atomizer of the diffuser.

Session rating 58 can provide a user feedback rating provided by the user. In some implementations, session rating may be correlated with scent information 52 by the application executed on remote device 22 and/or a server (e.g., server 26 in FIG. 21) to generate recommendations for other oils to try for a particular user.

Session notes 60 can provide the user with the option to add information about the session, such as notes about how different oils made the user feel during the session. Other notes may be left by the user, such as music listened to during the session or a particular mediation or breathing exercise used during the session, for example.

FIG. 25 illustrates an example session history graphical user interface displayed on user interface 28 of remote device 22 according to one or more embodiments. As shown in FIG. 25, session trends selection 62 can enable a user to select different periods, such as weekly, monthly, or yearly, to display information about the sessions during the period. In the example of FIG. 25, the user has selected “Week” for their session trends, and user interface 28 displays the number of sessions each week.

Recent sessions selection 64 enables the user to display information about recent sessions with the diffuser or with multiple diffusers. Session information 65 provides an example of information that may be displayed for a recent session, such as the scent of oil used, when the session occurred, and the duration of session.

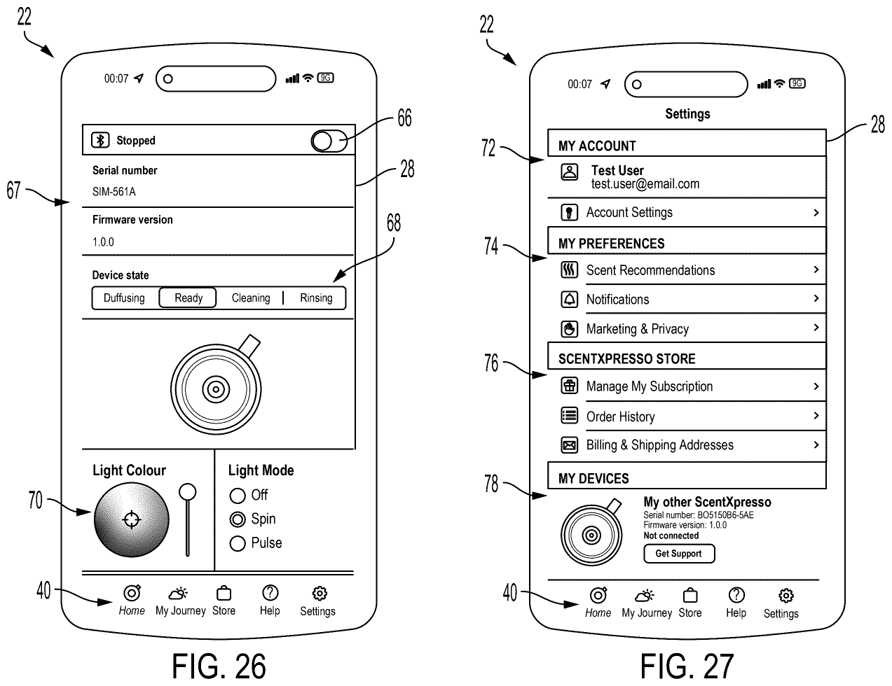

FIG. 26 illustrates an example device settings graphical user interface displayed on user interface 28 of remote device 22 according to one or more embodiments. As shown in FIG. 26, the device settings can include wireless interface control 66, device information 67, device state 68, and lighting control 70. Wireless interface control 66 can provide an enabling or disabling of the wireless communication of the diffuser, such as with the toggle switch shown in FIG. 26. Device information 67 can include information about the diffuser, such as its serial number and firmware version.

Device state 68 can indicate a current state or operating cycle for the diffuser. In the example of FIG. 26, the device state can indicate whether the diffuser is currently diffusing a scented vapor, is ready to operate, is performing a cleaning cycle, or is performing a rinse cycle, such as at the end of a diffusion cycle. In some implementations, a separate cleaning or descaling cycle may be initiated from the application. The application may provide guided instructions for a user to add a cleaning or decalcification material to the base liquid tank of the diffuser for the cleaning cycle to rinse the mixing chamber and diffuse from the atomizer. In some cases, a user may set a water hardness level using the application that may notify the user of when to perform a cleaning or decalcification cycle after a threshold amount of use of the particular diffuser.

Lighting control 70 can provide options for different lighting effects, such as for lighting ring 106 and/or side lights 113 and 115. In the example of FIG. 26, a color of the lights can be selected on a color map by the user, and a brightness of the lights can be set using a slider. In addition, a light mode to provide an optical movement feature can be selected, such as a spin mode where the sequence of lighting LEDs around light ring 106 provides a light spinning effect, or a pulse mode where the intensity of the lights is pulsed. In some implementations, a pulsing light mode may be used for a visual breathing instruction to the user or may be coordinated with a meditation or health application executed by remote device 22. In yet other examples, the optical movement feature may be coordinated with audio that is played by the remote device to synchronize the lighting effect with a rhythm or beats per minute of the audio, such as music or ambient sounds.

FIG. 27 illustrates an example user settings graphical user interface displayed on user interface 28 of remote device 22 according to one or more embodiments. As shown in FIG. 27, the user settings can include account information 72, user preferences 74, store interface 76, and device information 78. Account information 72 can include user information, such as the user's email address, and settings for the user's account, such as a password.

User preferences 74 can include recommendations for other scents to try based on the history of scents previously used, feedback from the user on scents, and/or feedback from other users, such as reviews. In addition, user preferences 74 can include notification settings for the application and privacy settings for the user.

Store interface 76 can include, for example, a subscription for purchasing oils, an order history for the user, and billing and shipping information for the user. In some implementations, store interface 76 may open a browser window for an online store.

Device information 78 can include information on one or more devices used or owned by the user. This may include a serial number for the diffuser, a firmware version currently installed on the diffuser, and a connection status. Device information 78 may also provide an option for obtaining technical support for the diffuser or the application.

Those of ordinary skill in the art will appreciate with reference to the present disclosure that other graphical user interfaces are possible that may display different information or in a different configuration than shown in FIGS. 22 to 27. For example, session notes 60, the remaining amount of oil left in a particular oil container, and/or scent recommendations may be omitted in some implementations.

### Example Processes

FIG. 28 is a flowchart for a controlled mixing process according to one or more embodiments. The process of FIG. 28 can be performed by, for example, circuitry 131 of diffuser 100 by processor(s) 133 executing firmware stored in memory 135 in FIG. 7. In this regard, circuitry 131 can, in some implementations, comprise a means for performing the functions of the controlled mixing process of FIG. 28.

In block 2802, an input is received to begin an operating cycle. The input may be received, for example, from a user pressing a button on the diffuser (e.g., central button 104) or may be wirelessly received from a remote device executing an application. In the example of FIG. 28, the operating cycle can refer to a session where scented mist or vapor is to be diffused from the diffuser.

In block 2804, the circuitry actuates a base liquid valve (e.g., base liquid valve 159 in FIG. 17) to dispense a controlled amount of base liquid from a base liquid tank (e.g., from an interior of base liquid tank 126) into a mixing chamber (e.g., mixing chamber cavity 144). The controlled amount of base liquid can be determined or calculated based on, for example, a duration set for the operating cycle, a scent strength set for the operating cycle, a volume of the mixing chamber, and/or a desired viscosity for a mixture of oil and base liquid. In some implementations, a channel between the base liquid valve and the mixing chamber is based, at least in part, on a drainage rate of the base liquid from the base liquid tank. For example, the amount of time that the base liquid valve is opened or in contact with an actuator of the diffuser can be controlled based on a predetermined drainage rate of base liquid from the base liquid tank.

In block 2806, the circuitry actuates an oil valve (e.g., oil valve 164) to dispense a controlled amount of oil from an oil container (e.g., oil container 16) that has been inserted into the diffuser. In some implementations, an amount of time or contact time for an actuator of the diffuser may be based on the controlled amount of oil to be dispensed. As with the base liquid, the controlled amount of oil can be determined or calculated based on, for example, a duration set for the operating cycle, a scent strength set for the operating cycle, a volume of the mixing chamber (e.g., preferably between four to eleven milliliters), and/or a desired viscosity for a mixture of oil and base liquid.

In block 2808, an atomizer of the diffuser (e.g., atomizer 102) is activated to atomize a mixture of the base liquid and the oil from the mixing chamber to an exterior of the diffuser. The circuitry may activate the atomizer by, for example, providing a voltage signal to a piezoelectric element of the atomizer that vibrates a mesh component of the atomizer and propels a vapor or mist from the mesh component to the exterior of the diffuser.

Notably, the atomized mixture is diffused in a direction that is substantially perpendicular to a gravitational direction in which gravity acts on the diffuser when the diffuser is in an operating orientation. As opposed to conventional diffusers that may diffuse a vapor in a more vertical direction, the diffuser in block 2808 can provide a more direct diffusion towards a user's face or upper body, enhancing the sensory experience by concentrating the aroma in a zone of heightened perceptual sensitivity. This directional delivery provides a distinct user experience advantage in personal care settings, such as during showers or meditation. As discussed above, the arrangement of the relatively small mixing chamber with a substantially horizontal atomizer that includes a mesh component that enables the mixture to pass through can facilitate a substantially horizontal diffusion of the mixture.

In addition, the use of an ultrasonic mesh atomizer introduces a visual mist effect, adding a multisensory dimension to the user experience. Unlike traditional nebulizer-based diffusers that produce little or no visible vapor, this method creates a more immersive feedback loop between scent and sight, reinforcing the perceived effectiveness of the diffusion process.

Furthermore, the relatively small mixing chamber and controlled dispensing of oil and base liquid avoids premature mixing of oil and base liquid. By dispensing each liquid on-demand into a smaller mixing chamber, the resulting vapor is fresher and more aromatic, which enhances scent strength and conserves essential oil.

Those of ordinary skill in the art will appreciate with reference to the present disclosure that other implementations of the process of FIG. 28 may differ. For example, the order of blocks 2804 and 2806 may be switched in other implementations, such that the oil is added to the mixing chamber before the base liquid. As another example variation, the performance of blocks 2804 and 2806 may alternate back and forth at a particular frequency to dispense the controlled amounts of oil and base liquid to the mixing chamber throughout a session.

FIG. 29 is a flowchart for a controlled amount calculation process according to one or more embodiments. The process of FIG. 29 can be performed by, for example, circuitry 131 of diffuser 100 by processor(s) 133 executing firmware stored in memory 135 in FIG. 7. In other implementations, the controlled amount calculation process of FIG. 29 may be partly performed by a processor executing a stored application at a remote device to calculate the indication of the controlled amount of oil and/or base liquid to dispense and then communicate the calculated indication(s) to circuitry of the diffuser. In this regard, circuitry 131 and/or a remote device processor and memory can, in some implementations, comprise a means for performing the functions of the controlled amount calculation process of FIG. 29.

The process of FIG. 29 may be used, in some implementations, to calculate a default indication of an amount of oil and/or base liquid to dispense for sessions, unless indicated otherwise by a user. In addition, the process of FIG. 29 may be used to change the controlled amount of oil and/or base liquid to be dispensed for one or more sessions in progress or before beginning the session.

In block 2902, the circuitry or processor calculates at least one of an indication of the controlled amount of oil to dispense and the controlled amount of base liquid to dispense based on at least one of a user input, a type of oil, and a viscosity of the mixture of base liquid and oil. For example, a user may use an application executing on a remote device to provide a desired scent strength and the processor of the remote device may calculate indications for both the time for the base liquid valve to be open and the time for the oil valve to be open, using for example, a lookup table. As another example, the diffuser may receive a user input from a user pressing a button on the diffuser (e.g., side button 112) to increase a scent strength for a current session. The circuitry of the diffuser may then calculate an indication of a controlled amount of oil to dispense in response to the user input.

In block 2904, the circuitry of the diffuser dispenses at least one of the controlled amount of oil and the controlled amount of base liquid based on the one or more calculated indications of the controlled amount(s). In some cases, the circuitry may wirelessly receive the calculated indication from a remote device. In other cases, the circuitry may calculate the indication. As discussed above for the controlled mixing process of FIG. 28, the circuitry of the diffuser may control one or more actuators (e.g., actuator 174 via motor assembly 189) to dispense the controlled amount of oil and/or base liquid.

Those of ordinary skill in the art will appreciate with reference to the present disclosure that other implementations of the process of FIG. 29 may differ. For example, the process of FIG. 29 may repeat throughout an operating cycle, such as where a user provides one or more inputs to increase or decrease a scent strength during the operating cycle.

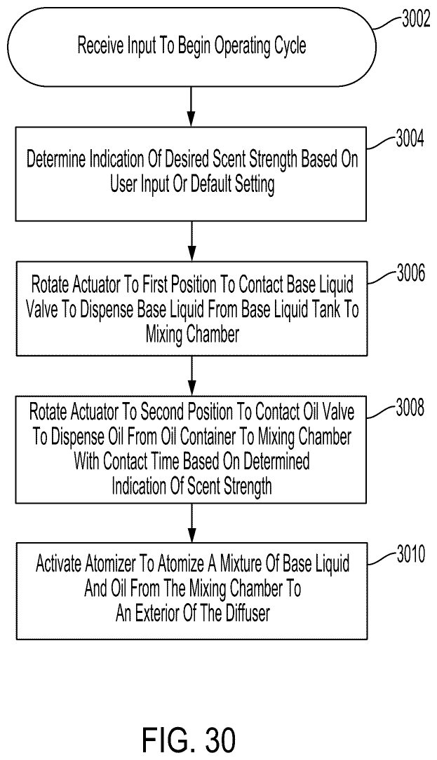

FIG. 30 is a flowchart for an operating cycle process according to one or more embodiments. The process of FIG. 30 can be performed by, for example, circuitry 131 of diffuser 100 by processor(s) 133 executing firmware stored in memory 135 in FIG. 7. In this regard, circuitry 131 can, in some implementations, comprise a means for performing the functions of the operating cycle process of FIG. 30.

In block 3002, the circuitry receives an input to begin an operating cycle. The input can include, for example, a user input from a button on the diffuser or a wirelessly received input, such as from a remote device.

In block 3004, the circuitry determines an indication of desired scent strength based on a user input or a default setting. The user input can include, for example, a user pressing a button on the diffuser to increase or decrease the scent strength or may include a scent strength set by the user via a graphical user interface of an application executed by a remote device. In some cases, the diffuser may have a default scent strength setting that is used unless a user input changes the default setting.

In block 3006, the circuitry rotates an actuator (e.g., actuator 174) to a first position to contact a base liquid valve to dispense base liquid from a base liquid tank of the diffuser to a mixing chamber of the diffuser. In some implementations, the circuitry may rotate the actuator via a motor assembly (e.g., motor assembly 189) that is controlled by the circuitry. As discussed above with reference to FIGS. 19 and 20, the circuitry may use a feedback sensor, such as a hall sensor to position the actuator into contact with the base liquid valve, such as by contacting a ball or other seal of the base liquid valve (e.g., ball 186 of base liquid valve 159 in FIG. 17).

In block 3008, the circuitry rotates the actuator to a second position to contact an oil valve to dispense oil from an oil container to a mixing chamber of the diffuser. The contact time of the actuator to the oil valve in block 3008 is based on the determined indication of scent strength from block 3004. In some implementations, the determined indication of desired scent strength from block 3004 can correspond to a ratio or concentration setting for the amount of oil dispensed for a given amount of base liquid dispensed, for example.

In some implementations, the circuitry may rotate the actuator via a motor assembly (e.g., motor assembly 189) that is controlled by the circuitry. The circuitry may use a feedback sensor, such as a hall sensor to position the actuator into contact with the oil valve, such as by contacting a ball or other seal of the oil valve (e.g., ball 166 of base liquid valve 159 in FIG. 17). The use of a single actuator in the example of FIG. 30 can facilitate a more efficient operation and design of the diffuser by not requiring separate actuators for the base liquid valve and the oil valve.

In block 3010, an atomizer of the diffuser (e.g., atomizer 102) is activated to atomize a mixture of the base liquid and the oil from the mixing chamber to an exterior of the diffuser. The circuitry may activate the atomizer by, for example, providing a voltage signal to a piezoelectric element of the atomizer that vibrates a mesh component of the atomizer and propels a vapor or mist from the mesh component to the exterior of the diffuser.

Those of ordinary skill in the art will appreciate with reference to the present disclosure that other implementations of the process of FIG. 30 may differ. For example, the order of contacting the base liquid valve and oil valve may be switched such that block 3008 is performed before block 3006 in some implementations. As another example variation, block 3006 or block 3008 may be performed concurrently with block 3010 to release more base liquid or more oil while the atomizer is active in block 3010. In addition, the actuator may be rotated back and forth between the first position and the second position during a single session at a determined frequency, or may be rotated to a third position, such as a home position or a venting position.

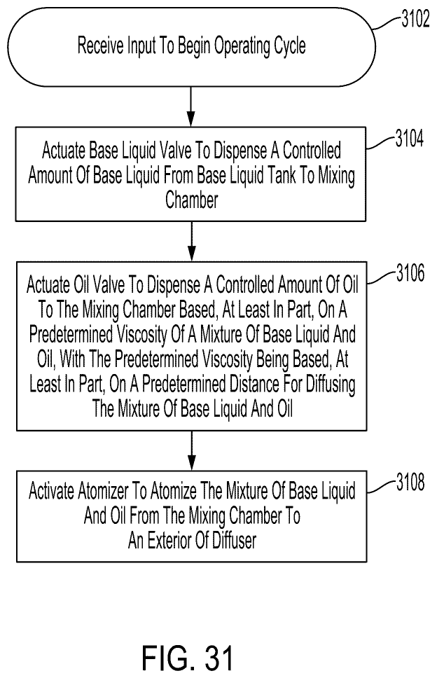

FIG. 31 is a flowchart for an operating cycle process according to one or more embodiments. The process of FIG. 31 can be performed by, for example, circuitry 131 of diffuser 100 by processor(s) 133 executing firmware stored in memory 135 in FIG. 7. In this regard, circuitry 131 can, in some implementations, comprise a means for performing the functions of the operating cycle process of FIG. 31.

In block 3102, the circuitry receives an input to begin an operating cycle. The input can include, for example, a user input from a button on the diffuser or a wirelessly received input, such as from a remote device.

In block 3104, the circuitry actuates a base liquid valve (e.g., base liquid valve 159 in FIG. 17) to dispense a controlled amount of base liquid from a base liquid tank (e.g., base liquid tank 126) into a mixing chamber (e.g., mixing chamber cavity 144). The controlled amount of base liquid can be determined or calculated based on, for example, a duration set for the operating cycle, a scent strength set for the operating cycle, a volume of the mixing chamber, and/or a desired viscosity for a mixture of oil and base liquid. In some implementations, a channel between the base liquid valve and the mixing chamber is based, at least in part, on a drainage rate of the base liquid from the base liquid tank. For example, the amount of time that the base liquid valve is opened or in contact with an actuator of the diffuser can be controlled based on a predetermined drainage rate of base liquid from the base liquid tank.

In block 3106, the circuitry actuates an oil valve (e.g., oil valve 164) to dispense a controlled amount of oil to the mixing chamber based, at least in part, on a predetermined viscosity of a mixture of base liquid and oil. The predetermined viscosity is based, at least in part, on a predetermined distance for diffusing the mixture of base liquid and oil. For example, a user may increase a target diffusion distance via an application executed on a remote device. The remote device may provide this setting to the circuitry of the diffuser, which may decrease the amount of oil to be diffused during the session to provide a lower viscosity for the mixture that will diffuse farther than a higher viscosity of the mixture that would include more oil. The circuitry may consider, for example, the base liquid being used (e.g., a particular hydrosol versus water) and/or the type of oil being used, which may contribute differently to the viscosity of the mixture.

In Ir block 3108, an atomizer of the diffuser (e.g., atomizer 102) is activated to atomize the mixture of the base liquid and the oil from the mixing chamber to an exterior of the diffuser. The circuitry may activate the atomizer by, for example, providing a voltage signal to a piezoelectric element of the atomizer that vibrates a mesh component of the atomizer and propels a vapor or mist from the mesh component to the exterior of the diffuser.

Those of ordinary skill in the art will appreciate with reference to the present disclosure that other implementations of the process of FIG. 31 may differ. For example, the order of dispensing the base liquid and the oil may be switched such that block 3106 is performed before block 3104 in some implementations. As another example variation, block 3104 or block 3106 may be performed concurrently with block 3108 to release more base liquid or more oil while the atomizer is active in block 3108. In addition, the actuation of the oil valve and the base liquid valve may be alternated between each other multiple times during a single session, such as at a particular frequency of time during the session to maintain enough of the mixture in the mixing chamber during the session.

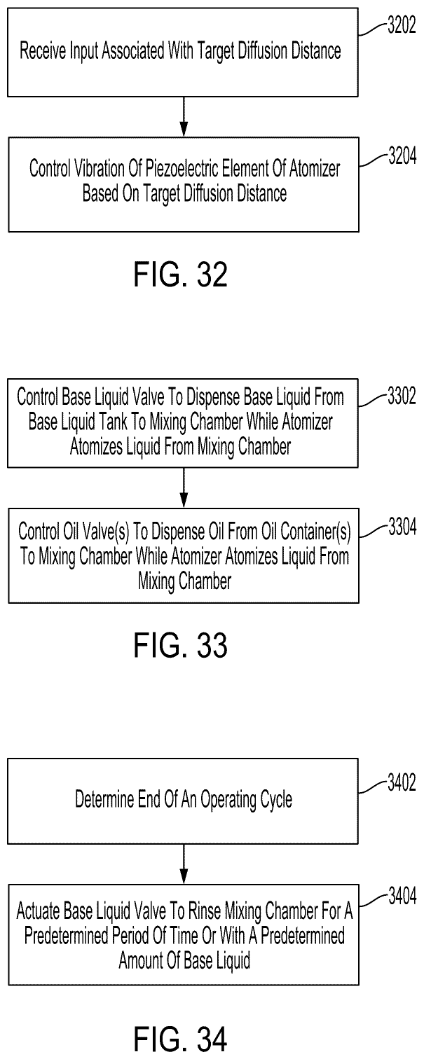

FIG. 32 is a flowchart for a diffusion distance control process according to one or more embodiments. The process of FIG. 32 can be performed by, for example, circuitry 131 of diffuser 100 by processor(s) 133 executing firmware stored in memory 135 in FIG. 7. In this regard, circuitry 131 can, in some implementations, comprise a means for performing the functions of the diffusion distance control process of FIG. 32.

In block 3202, an input is received by the circuitry that is associated with a target diffusion distance. For example, an application executed by a remote device may receive an input via a diffuser spread control (e.g., diffuser spread control 56 in FIG. 24) that may correspond to a particular target diffusion distance, such as 50 centimeters from the diffuser. This input may be wirelessly transmitted to the circuitry of the diffuser. In another example, a user may press one of the buttons on the diffuser (e.g., a side button 112 or 114) to increase or decrease the target diffusion distance.

In block 3204, the circuitry controls the vibration of a piezoelectric element of an atomizer of the diffuser (e.g., piezoelectric element 132 in FIG. 10) based on the target diffusion distance. In some implementations, the circuitry may set a voltage or power to supply to the piezoelectric element that corresponds to a particular frequency of vibration imparted onto a mesh component (e.g., mesh component 116) of the atomizer. In this regard, a higher vibration of the mesh component can result in a farther diffusion of the mixture from the diffuser.

Those of ordinary skill in the art will appreciate with reference to the present disclosure that other implementations of the process of FIG. 32 may differ. For example, the diffuser may use a default diffusion distance, such that the vibration of the piezoelectric element in block 3204 may not be based on a received input, but instead, may be based on a predetermined setting stored in a memory of the circuitry.

FIG. 33 is a flowchart for an ongoing valve control process according to one or more embodiments. The process of FIG. 33 may be performed after an operating cycle of the diffuser has already been initiated. For example, the ongoing valve control process of FIG. 33 can be performed in conjunction with any of the processes of FIGS. 28 to 32 discussed above. The process of FIG. 33 can be performed by, for example, circuitry 131 of diffuser 100 by processor(s) 133 executing firmware stored in memory 135 in FIG. 7. In this regard, circuitry 131 can, in some implementations, comprise a means for performing the functions of the ongoing valve control process of FIG. 33.

In block 3302, the circuitry controls a base liquid valve to dispense base liquid from a base liquid tank to a mixing chamber while an atomizer atomizes liquid from the mixing chamber. In some cases, the liquid being atomized when the base liquid valve is controlled can be a mixture resulting from the initiation of a diffusion operating cycle using a default setting or user provided setting indicating a concentration of oil to base liquid (i.e., scent strength setting). In other cases, the liquid being atomized when the base liquid valve is controlled may be primarily base liquid following a rinse cycle or a startup of the operating cycle. In some implementations, the base liquid valve can be controlled to refill the mixing chamber or otherwise maintain a baseline level of liquid in the mixing chamber after diffusing liquid via the atomizer. The control of the base liquid valve may be, for example, through the use of an actuator or motor. In some implementations, the control of the base liquid valve may be based on an open time for the valve.

In block 3304, the circuitry controls one or more oil valves to dispense oil from one or more respective oil containers while the atomizer atomizes liquid from the mixing chamber. As discussed above, some embodiments may include mixing different oils in particular ratios or concentrations and may therefore involve individually controlling the amount of oil dispensed for each oil.

In some cases, the liquid being atomized when the oil valve(s) is controlled can be a mixture resulting from the initiation of a diffusion operating cycle using a default setting or user provided setting indicating a concentration of oil to base liquid (i.e., scent strength setting). In other cases, the liquid being atomized when the oil valve(s) is controlled may be primarily base liquid following a rinse cycle or a startup of the operating cycle. In some implementations, the one or more oil valves can be controlled to maintain the concentration of oil(s) in a mixture of base liquid and oil in the mixing chamber. The control of the oil valve(s) may be, for example, through the use of one or more actuators or motors. In some implementations, the control of the oil valve(s) may be based on an open time for the valve(s).

The ongoing control of valves, as in the process of FIG. 33, can provide a controlled, on-demand mixing of oil and base liquid, which ensures freshness of scent during use and avoids early evaporation or oil degradation. In this regard, a user input, such as from a button on the diffuser, can dynamically change the strength of the scent being diffused in near real-time due to the smaller size of the mixing chamber and the ongoing control of the base liquid valve and the oil valve(s). In contrast, conventional ultrasonic diffusers typically require that the user dump out a larger volume of oil and base liquid from a reservoir and manually refill base liquid and carefully add oil drops to change the concentration of oil or imprecisely add more drops to an existing mixture of unknown concentration.

Those of ordinary skill in the art will appreciate with reference to the present disclosure that other implementations of the ongoing valve control process of FIG. 33 may differ. For example, the order of blocks 3302 and 3304 may be switched. In addition, of ordinary skill in the art will appreciate with reference to the present disclosure that the performance of blocks 3302 and 3304 may by repeated many times throughout a single operating cycle or session.

FIG. 34 is a flowchart for a rinse process according to one or more embodiments. The process of FIG. 34 can be performed by, for example, circuitry 131 of diffuser 100 by processor(s) 133 executing firmware stored in memory 135 in FIG. 7. In this regard, circuitry 131 can, in some implementations, comprise a means for performing the functions of the rinse process of FIG. 34.

In block 3402, the circuitry determines the end of an operating cycle or session of using the diffuser to diffuse a scented vapor or mist. In some cases, the end of the operating cycle can be determined by the expiration of a duration time received from a remote device. The duration of the operating cycle may be set for the session via a graphical user interface of an application executed by a remote device (e.g., via duration control 50 in FIG. 24). In other cases, the end of the operating cycle may be determined by receiving an input via a button of the diffuser (e.g., central button 104). In yet other cases, the end of the operating cycle may be determined by the expiration of a default session time stored in a memory of the circuitry (e.g., eight minutes).

In block 3404, the circuitry actuates a base liquid valve of the diffuser to rinse a mixing chamber of the diffuser for a predetermined period of time or with a predetermined amount of base liquid. For example, the circuitry may actuate a valve such as base liquid valve 159 in FIG. 17 via actuator 174 and motor assembly 189 for a predetermined ten seconds or to provide a predetermined amount of base liquid, such as 100 ml, for example. The atomizer may continue to diffuse mist or vapor without oil to clean the mesh component and the mixing chamber. This can reduce cross contamination of oils between sessions and prevent the oils from reducing the efficiency of components such as the atomizer by preventing the oil from clogging small holes in the mesh component. During the rinse cycle, the diffuser may also provide an indication such as by changing a color of the lighting or using a lighting sequence to create a spinning appearance, for example.

Those of ordinary skill in the art will appreciate with reference to the present disclosure that other implementations of the rinse process of FIG. 34 may differ. For example, other implementations may also perform the rinse process before initiating an operating cycle.

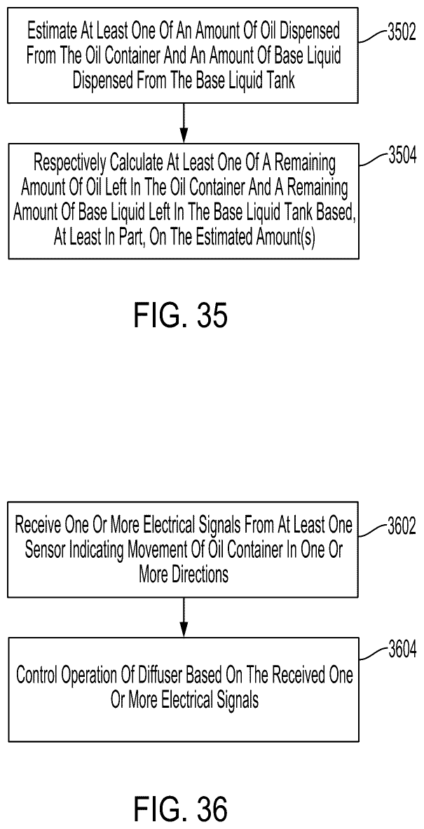

FIG. 35 is a flowchart for a remaining liquid determination process according to one or more embodiments. The process of FIG. 35 can be performed by, for example, circuitry 131 of diffuser 100 by processor(s) 133 executing firmware stored in memory 135 in FIG. 7. In other implementations, the remaining liquid determination process of FIG. 35 may be partly performed by a processor executing a stored application at a remote device to determine the amount of remaining liquid and then communicate the determined amount to circuitry of the diffuser. In this regard, circuitry 131 and/or a remote device processor and memory can, in some implementations, comprise a means for performing the functions of the remaining liquid determination process of FIG. 35.

In block 3502, the circuitry estimates at least one of an amount of oil dispensed from the oil container and an amount of base liquid dispensed from the base liquid tank. In some implementations, the circuitry may use a time that an actuator is in a position corresponding to an opening of a valve for the oil or the base liquid (e.g., the first position or the second position discussed above for FIGS. 16 to 18) to estimate the amount of oil or base liquid dispensed. Each of the oil valve and the base liquid valve may be associated with a predetermined flow rate that can be multiplied with the time that the valve is open to estimate the dispensed amount of liquid in some implementations.

In block 3504, at least one of a remaining amount of oil and a remaining amount of base liquid is calculated based, at least in part, on the amount(s) estimated in block 3502. In some implementations, a current volume of oil in an oil container may be stored in a memory of the circuitry of the diffuser (e.g., memory 135 of circuitry 131 in FIG. 17), a memory of a remote device (e.g., displayed as remaining amount indicator 46 in FIG. 23 or oil information 32 in FIG. 22), or a memory of a receptacle (e.g., memory 171 of receptacle tag 163 in FIG. 13). The estimated amount of oil dispensed from block 3502 may then be subtracted from the stored current volume to provide a new current volume of oil that may then replace the old current volume stored in the memory. As discussed above, the oil container can include a unique identifier, such as a QR code or tag (e.g., QR code 20 or tag 18 in FIG. 13) that enables the circuitry or the remote device to update the current volume of oil for a particular oil container.

As another example, the base liquid tank of the diffuser may have a level sensor that indicates when the base liquid tank is full of liquid or at certain levels such as every 10% increment of volume of the full capacity. A similar calculation may be performed by the circuitry of the diffuser or by a remote device to more closely determine a remaining amount of base liquid in the base liquid tank.

The remaining amount(s) may be used to alert the user when a low level of liquid has been reached that would not provide enough liquid for an operating cycle and/or a rinse cycle, such as through lighting on the diffuser or a notification on a graphical user interface executed at the remote device. Indications of the remaining amount(s) (e.g., a percentage left) may also be displayed as a status for the base liquid tank or oil container in a graphical user interface of the remote device.

Those of ordinary skill in the art will appreciate with reference to the present disclosure that other implementations of the remaining liquid determination process of FIG. 35 may differ. For example, other implementations may only determine the remaining oil and may instead rely on a level sensor in the base liquid tank (e.g., base liquid level sensor 145 in FIG. 17) to determine the remaining amount of base liquid.

FIG. 36 is a flowchart for a diffuser control process according to one or more embodiments. The process of FIG. 36 can be performed by, for example, circuitry 131 of diffuser 100 by processor(s) 133 executing firmware stored in memory 135 in FIG. 7. In this regard, circuitry 131 can, in some implementations, comprise a means for performing the functions of the diffuser control process of FIG. 36.

In block 3602, the circuitry receives one or more electrical signals from at least one sensor indicating movement of an oil container in one or more directions. With reference to FIG. 11 discussed above, an oil container inserted into receptacle 156 may be moved by a user as a joystick to contact a sensor, such as contact sensor 139. The contact sensor may use, for example, a gimbal assembly to convert the detected movement into one or more electrical signals provided to the circuitry.

In block 3604, the circuitry controls operation of the diffuser based on the one or more electrical signals received in block 3602. For example, the movement of the oil container in one direction may cause the circuitry to increase the concentration of oil dispensed to the mixing chamber or may increase the remaining time for a current session. The movement of the oil container in the opposite direction may cause the circuitry to decrease the concentration of oil dispensed to the mixing chamber or may decrease the remaining time for the current session.

Those of ordinary skill in the art will appreciate with reference to the present disclosure that other implementations may differ. For example, the diffuser control process of FIG. 36 may be omitted in implementations where the diffuser may instead be controlled by buttons on the diffuser, such as side buttons 112 and 114.

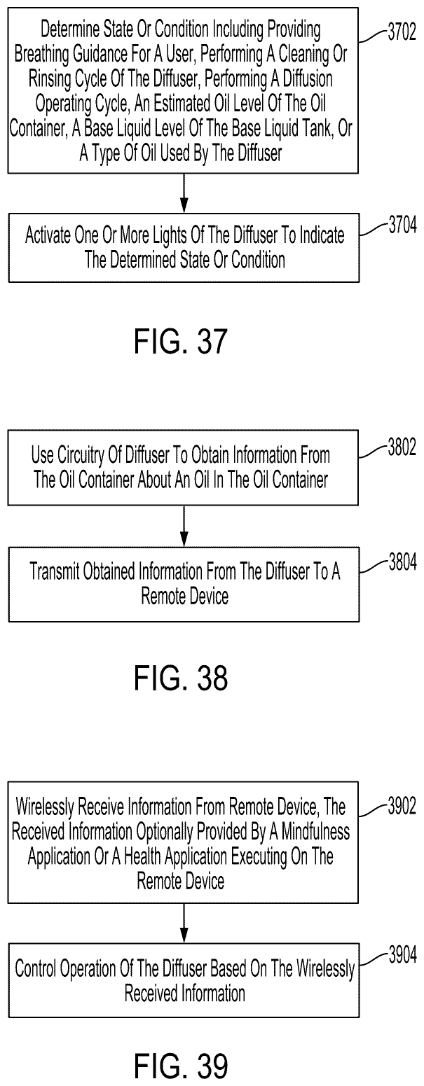

FIG. 37 is a flowchart for a diffuser state or condition indication process using one or more lights of the diffuser according to one or more embodiments. The process of FIG. 37 can be performed by, for example, circuitry 131 of diffuser 100 by processor(s) 133 executing firmware stored in memory 135 in FIG. 7. In this regard, circuitry 131 can, in some implementations, comprise a means for performing the functions of the diffuser state or condition indication process of FIG. 37.

In block 3702, the circuitry determines a state or condition of the diffuser. The state or condition can include providing breathing guidance for a user, performing a cleaning or rinsing cycle of the diffuser, performing a diffusion operating cycle, an estimated oil level of the oil container, a base liquid level of the base liquid tank, or a type of oil used by the diffuser. In the example of breathing guidance, a remote device may communicate one or more settings to the circuitry of the diffuser, such as a target breathing rate that the circuitry may use to activate or pulse the intensity of one or more lights of the diffuser.

In block 3704, the circuitry activates one or more lights of the diffuser (e.g., lighting ring 106 and/or side lights 113 and 115) to indicate the determined state or condition. The activation can include, for example, changing the color of the lights or sequencing the activation of the lights to create an effect, such as a flashing effect or a spinning effect. In some implementations, the determined state or condition may also be indicated as a notification or other visual or audible notification on a remote device.

Those of ordinary skill in the art will appreciate with reference to the present disclosure that other implementations may differ. For example, a different condition or state may trigger the activation of lights of the diffuser, such as the playing of music through an application executed by a remote device that may instruct the circuitry of the diffuser to control lighting to operate based on a rhythm of the music being played.

FIG. 38 is a flowchart for an oil container information collection process according to one or more embodiments. The process of FIG. 38 can be performed by, for example, circuitry 131 of diffuser 100 by processor(s) 133 executing firmware stored in memory 135 in FIG. 7. In this regard, circuitry 131 can, in some implementations, comprise a means for performing the functions of the oil container information collection process of FIG. 38.

In block 3802, the circuitry of the diffuser obtains information from an oil container about the oil in the oil container. In some implementations, this can include using a sensor of the diffuser (e.g., sensor 141 in FIG. 11) to read information from the oil container, such as from a QR code or a radio tag on the oil container. The information about the oil can include, for example, a brand or manufacturer of the oil, a scent of the oil, an expiration date for the oil, or a manufacture date for the oil.

In block 3804, the information obtained by the circuitry of the diffuser is transmitted to a remote device, such as by using a wireless interface (e.g., wireless interface 137 in FIG. 7). The information about the oil may then be used by an application executing on the remote device to display the information on a graphical user interface, as with the examples of FIGS. 22 to 24 discussed above with oil information 32, oil container details 44, and scent information 52. The use of circuitry of the diffuser, such as with sensor 141 and circuitry 131 of diffuser 100, to obtain information from an oil container can enable an application executing on a remote device to provide the information on a currently used or currently inserted oil container to the user.

Those of ordinary skill in the art will appreciate with reference to the present disclosure that other implementations may differ. For example, a QR code or radio tag may be read by the remote device without involving the circuitry of the diffuser in some implementations.

FIG. 39 is a flowchart for a diffuser control process via a remote device according to one or more embodiments. The process of FIG. 39 can be performed by, for example, circuitry 131 of diffuser 100 by processor(s) 133 executing firmware stored in memory 135 in FIG. 7. In this regard, circuitry 131 can, in some implementations, comprise a means for performing the functions of the diffuser control process of FIG. 39.

In block 3902, the circuitry wirelessly receives information from a remote device. The information may be received via a wireless interface of the circuitry, such as wireless interface 137 in the example of FIG. 7, which can include a Wi-Fi interface or a Bluetooth interface. In some implementations, the information may be provided by a mindfulness application (e.g., a meditation application) or a health application executing on the remote device. Such information can include, for example, a target breathing rate for a user, a Heart Rate Variability (HRV) of a user, a pulse of a user, and/or a desired goal for the user (e.g., relaxation before a particular bedtime or increased alertness after waking).

In block 3904, the circuitry controls operation of the diffuser based on the wirelessly received information from block 3902. For example, lights of the diffuser, a scent strength (e.g., the concentration of oil added to the mixing chamber), a duration for a session, the initiation of an operating cycle or a cleaning cycle, or a frequency of vibrating a piezoelectric element of an atomizer (e.g., based on a received target diffusion distance) may be controlled based on the information received from the remote device.

Those of ordinary skill in the art will appreciate with reference to the present disclosure that other implementations may differ. For example, some implementations of the diffuser may not include wireless communication capability and may communicate with a remote device through a wired connection or may not communicate with a remote device at all.

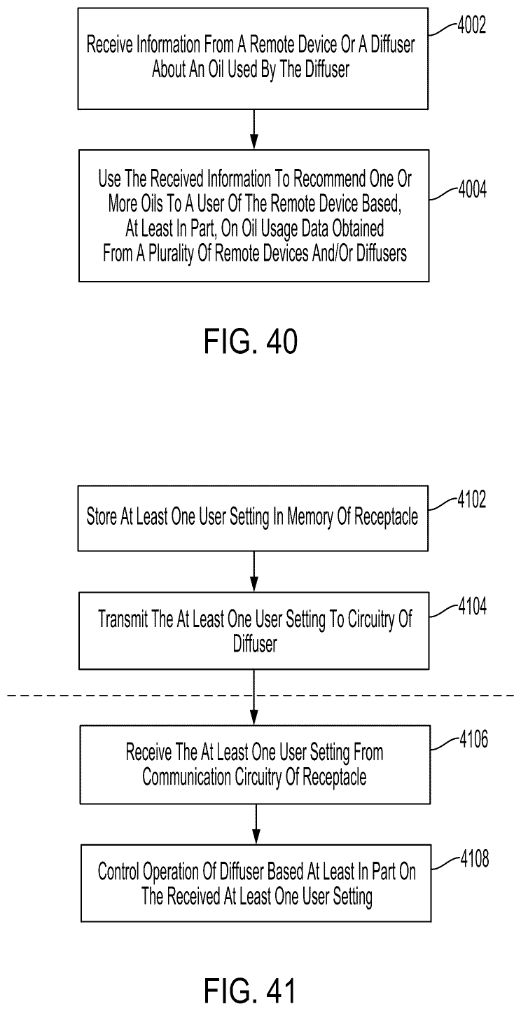

FIG. 40 is a flowchart for an oil recommendation process according to one or more embodiments. The process of FIG. 40 can be performed by, for example, one or more processors and memories of a server, such as server 26 in FIG. 21. In this regard, server 26 can, in some implementations, comprise a means for performing the functions of the oil recommendation process of FIG. 40.

In block 4002, information is received by the server from a diffuser or from a remote device, such as a smartphone, a tablet, or laptop, about an oil used by a diffuser. The information may include, for example, a rating of a scent or of a session that was provided by a user in an application executed by the remote device (e.g., via session rating 58 or session notes 60 in FIG. 24). In another example, the diffuser may collect information about an oil used by the diffuser, such as the scent or brand of the oil, using a sensor of the diffuser (e.g., sensor 141 in FIG. 11 or receptacle tag 163 in FIG. 13). The information may be received by the server via a network (e.g., network 24), such as the internet.

In block 4004, the server uses the received information to recommend one or more oils to a user of a remote device based, at least in part, on oil usage data obtained from a group of devices that includes at least one of remote devices and diffusers. In some implementations, the server may collect oil usage data including ratings on different oils and recommend an oil in a particular category of oils (e.g., oils for relaxation or oils for alertness). In some implementations, the server may use an AI model that may be trained using information collected from remote devices 22 for many users, such as geographical locations of the users, ages or sexes of the users, and/or ratings provided by the user and by other users for a variety of different scents. The recommendation can be sent by the server via the network back to a remote device associated with the user. In some cases, the recommendation may appear in a store access portion of a graphical user interface of the application executed by the user's remote device (e.g., store interface 76 in FIG. 27). In other cases, the recommendation may appear as a notification in a graphical user interface of the application.

Those of ordinary skill in the art will appreciate with reference to the present disclosure that other implementations of the oil recommendation process of FIG. 40 may differ. For example, the application executed by the remote device may instead provide the recommendation of block 4004, rather than a server. In such cases, the application may be preconfigured or updated via the network to provide recommendations, such as for other oils in the same category of an oil being used by a diffuser in communication with the remote device, without involving a separate server.

FIG. 41 is a flowchart for a user setting communication process according to one or more embodiments. The first part of the process including blocks 4102 and 4104 above the dashed line in FIG. 41 may be performed by circuitry of a receptacle, such as receptacle tag 163 in FIG. 13, and the second part of the process including blocks 4106 and 4108 below the dashed line may be performed by other circuitry of the diffuser, such as circuitry 131 in FIG. 7. In this regard, receptacle tag 163 and circuitry 131 can, in some implementations, comprise a means for performing the functions of the user setting communication process of FIG. 41.

In block 4102, at least one user setting is stored in a memory of a receptacle. The receptacle may be removable from the diffuser, such that a user can secure an oil container into the receptacle when it is removed from the diffuser. With reference to FIG. 13, the user setting(s) may be stored in memory 171 of receptacle tag 163. In some implementations, the user setting(s) can be provided via a wireless communication with a remote device executing an application using communication circuitry of the receptacle tag (e.g., communication circuitry 173 in FIG. 13). The user settings may include, for example, a preferred session duration for the user, a preferred diffusion distance for the user, and/or a preferred lighting configuration for the user.

In block 4104, the at least one user setting is transmitted from the receptacle to the circuitry of the diffuser. In some implementations, this may be performed by communication circuitry of the receptacle tag wirelessly communicating with wireless interface circuitry of the diffuser (e.g., wireless interface 137 in FIG. 7). In other implementations, the at least one user setting may be transmitted via an electrical connection between the circuitry of the diffuser and the receptacle tag, such as through a physical contact to the removable receptacle when it is inserted into the diffuser.

In block 4106, the at least one user setting is received by the circuitry of the diffuser (e.g., circuitry 131 in FIG. 7) from the communication circuitry of the receptacle. The circuitry of the diffuser may store the received user setting(s) in a memory of the circuitry for use during a session when the receptacle is inserted into the diffuser.

In block 4108, the operation of the diffuser is controlled based, at least in part, on the received user setting(s). This can provide a relatively seamless way of transferring a particular user's settings to the diffuser since the receptacle may be associated with the particular user. In this regard, the user settings for different users of the same diffuser may be different and their receptacles can cause the diffuser to operate differently (e.g., with different default session durations, different default diffusion distances, and/or different default lighting schemes) depending on which receptacle is inserted into the diffuser. In some implementations, the user setting(s) transmitted to the circuitry of the diffuser may be overridden by settings that may be provided by a user input of the diffuser (e.g., central button 104 and/or side buttons 112 and 114) and/or by an application on a remote device in communication with the diffuser.

The foregoing diffusers and processes can provide many advantages over conventional diffusers. For example, the ongoing dispensing of oil during a session can maintain a stronger scent throughout a session as compared to a user manually adding drops to a water reservoir. In this regard, the disclosed diffusers and processes can provide a real-time, controlled ratio of oil to base liquid to provide a particular scent strength that may be dynamically changed by a user during a session. In addition, the horizontal diffusion of the disclosed diffusers can diffuse a more concentrated scent toward the user's head in smaller spaces, such as in a shower. The controlled dispensing of oil into a smaller mixing chamber can also conserve the use of typically expensive essential oils so that oil is not wasted by adding too much for a session or by accidently spilling the oil.

Other advantages of the disclosed diffusers and processes over conventional diffusers include automatic rinsing or guided cleaning of the mixing chamber and atomizer to improve the long-term effectiveness of the diffuser and reduce cross-contamination of oils from one session to the next. In addition, the disclosed diffusers provide a quieter operation by not using a fan, which can be particularly noticeable in a small space or when a user is in close proximity to the diffuser. The disclosed diffusers and processes also provide for improved user interaction with the diffuser through remote devices, such as a smartphone.

### OTHER EMBODIMENTS

Those of ordinary skill in the art will appreciate that the various illustrative logical blocks, modules, and processes described in connection with the examples disclosed herein may be implemented as electronic hardware, computer software, or combinations of both. Furthermore, the foregoing processes can be embodied on a computer readable medium, which causes processor or controller circuitry to perform or execute certain functions.

To clearly illustrate this interchangeability of hardware and software, various illustrative components, blocks, and modules have been described above generally in terms of their functionality. Whether such functionality is implemented as hardware or software depends upon the particular application and design constraints imposed on the overall system. Those of ordinary skill in the art may implement the described functionality in varying ways for each particular application, but such implementation decisions should not be interpreted as causing a departure from the scope of the present disclosure.

The various illustrative logical blocks, units, modules, processor circuitry, and controller circuitry described in connection with the examples disclosed herein may be implemented or performed with a general purpose processor, a DSP, an ASIC, an FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. Processor or controller circuitry may also be implemented as a combination of computing devices, e.g., a combination of a DSP and a microprocessor, a plurality of microprocessors, an SoC, one or more microprocessors in conjunction with a DSP core, or any other such configuration.

The activities of a method or process described in connection with the examples disclosed herein may be embodied directly in hardware, in a software module executed by processor or controller circuitry, or in a combination of the two. The steps of the method or algorithm may also be performed in an alternate order from those provided in the examples. A software module may reside in RAM memory, flash memory, ROM memory, EPROM memory, EEPROM memory, registers, hard disk, removable media, optical media, or any other form of storage medium known in the art. An exemplary storage medium is coupled to processor or controller circuitry such that the processor or controller circuitry can read information from, and write information to, the storage medium. In the alternative, the storage medium may be integral to processor or controller circuitry. The processor or controller circuitry and the storage medium may reside in an ASIC or an SoC.

The foregoing description of the disclosed example embodiments is provided to enable any person of ordinary skill in the art to make or use the embodiments in the present disclosure. Various modifications to these examples will be readily apparent to those of ordinary skill in the art, and the principles disclosed herein may be applied to other examples without departing from the scope of the present disclosure. The described embodiments are to be considered in all respects only as illustrative and not restrictive. In addition, the use of language in the form of “at least one of A and B” in the following claims should be understood to mean “only A, only B, or both A and B.”

What is claimed is:

1. A diffuser, comprising:
a base liquid tank configured to hold a base liquid;
a receptacle configured to receive an oil container;
a mixing chamber;
an atomizer configured to atomize liquid from the mixing chamber to an exterior of the diffuser;
a base liquid valve configured to dispense base liquid from the base liquid tank to the mixing chamber while the atomizer atomizes liquid from the mixing chamber; and
an oil valve configured to dispense oil from the oil container to the mixing chamber while the atomizer atomizes liquid from the mixing chamber.

2. The diffuser of claim 1, wherein a surface of the atomizer forms part of an exterior surface of the diffuser, and wherein the atomizer is further configured to diffuse the atomized liquid from at least a portion of the surface directly to air that is external to the diffuser.

3. The diffuser of claim 1, wherein the atomizer is further configured to diffuse the atomized liquid in a direction substantially perpendicular to a gravitational direction in which gravity acts on the diffuser when the diffuser is in an operating orientation.

4. The diffuser of claim 1, wherein the atomizer includes a mesh component at least partially surrounded by a piezoelectric element configured to vibrate the mesh component.

5. The diffuser of claim 1, wherein the atomizer includes a first removable mesh component having a first mesh pattern that is interchangeable with a second removable mesh component having a second mesh pattern, and wherein the first mesh pattern and the second mesh pattern are configured to diffuse the atomized liquid in different diffusion ranges when operating in the atomizer.

6. The diffuser of claim 1, wherein the atomizer further includes a piezoelectric element configured to vibrate the mesh component; and
wherein the diffuser further comprises circuitry configured to:
control a vibration frequency of the piezoelectric element based on a target diffusion distance for the atomized liquid.

7. The diffuser of claim 1, further comprising:
an air vent fluidly connecting at least one of the mixing chamber and the base liquid tank to the exterior of the diffuser, wherein the air vent relieves an air pressure in at least one of the mixing chamber and the base liquid tank.

8. The diffuser of claim 1, wherein the volume of the mixing chamber is within the range of four milliliters to eleven milliliters.

9. The diffuser of claim 1, wherein the atomizer includes a mesh component that forms part of the mixing chamber.

10. The diffuser of claim 1, further comprising circuitry configured to control operation of the diffuser; and
wherein the receptacle is removable from the diffuser and includes:
a memory configured to store at least one user setting associated with a user of the diffuser; and
communication circuitry configured to transmit the at least one user setting to the circuitry; and
wherein the circuitry is further configured to:
receive the at least one user setting from the communication circuitry of the receptacle; and
control operation of the diffuser based at least in part on the received at least one user setting.

11. The diffuser of claim 1, further comprising:
at least one actuator configured to dispense a controlled amount of base liquid from the base liquid valve and to dispense a controlled amount of oil from the oil valve.

12. The diffuser of claim 1, further comprising:
an actuator configured to be rotatably moved between at least a first position and a second position; and
wherein, in the first position, the actuator contacts the base liquid valve to dispense base liquid from the base liquid tank, and in the second position, the actuator contacts the oil valve to dispense oil from the oil container.

13. The diffuser of claim 1, wherein the amount of oil dispensed from the oil valve is based, at least in part, on a predetermined viscosity of a mixture of base liquid and oil, and wherein the predetermined viscosity is based, at least in part, on at least one of a predetermined distance for diffusing the atomized mixture of base liquid and oil from the atomizer and a volume of the mixing chamber.

14. The diffuser of claim 1, further comprising:
a channel between the base liquid valve and the mixing chamber, wherein an angle of the channel relative to a gravitational direction is based, at least in part, on a drainage rate of the base liquid from the base liquid tank.

15. The diffuser of claim 1, wherein the receptacle includes at least one sensor configured to receive a mechanical input from movement of the oil container; and
wherein the diffuser further comprises circuitry configured to:
receive one or more electrical signals from the at least one sensor indicating movement of the oil container in one or more directions; and
control operation of the diffuser based on the received one or more electrical signals.

16. The diffuser of claim 1, further comprising:
circuitry configured to:
determine an end of an operating cycle of the diffuser; and
in response to determining the end of the operating cycle, control actuation of the base liquid valve to rinse the mixing chamber for a predetermined period of time or with a predetermined amount of base liquid.

17. The diffuser of claim 1, further comprising a first removable face plate on a face side of the diffuser that is interchangeable with a second removable face plate, wherein the atomizer is further configured to atomize liquid from the face side, and wherein at least one of the appearance and a lighting arrangement of the first removable face plate differs from the second removable face place.

18. The diffuser of claim 1, further comprising one or more liquid level sensors configured to provide one or more respective electrical signals indicating a respective liquid level in at least one of the base liquid tank and the mixing chamber.

19. A diffuser, comprising:
a base liquid tank;
a receptacle configured to receive an oil container;
a mixing chamber;
a base liquid valve configured to dispense base liquid from the base liquid tank to the mixing chamber;
an oil valve configured to dispense oil from the oil container to the mixing chamber; and
an atomizer configured to atomize a mixture of base liquid and oil from the mixing chamber to an exterior of the diffuser in a direction substantially perpendicular to a gravitational direction in which gravity acts on the diffuser when the diffuser is in an operating orientation.

20. The diffuser of claim 19, wherein a surface of the atomizer forms part of an exterior surface of the diffuser, and wherein the atomizer is further configured to diffuse the atomized liquid from at least a portion of the surface directly to air that is external to the diffuser.

21. The diffuser of claim 19, wherein the atomizer includes a mesh component, and wherein an interior surface of the mesh component forms part of the mixing chamber.

22. The diffuser of claim 20, wherein the surface of the atomizer is formed by a first removable mesh component having a first mesh pattern that is interchangeable with a second removable mesh component having a second mesh pattern, and wherein the first mesh pattern and the second mesh pattern are configured to diffuse the atomized mixture of base liquid and oil in different diffusion ranges when operating in the atomizer.

23. The diffuser of claim 22, wherein each of the first mesh component and the second mesh component includes an electronically readable identifier, and wherein the diffuser further comprises circuitry configured to:
receive an input indicating use of the first mesh component or the second mesh component in the atomizer; and
change operation of the diffuser based on the received input.

24. The diffuser of claim 19, wherein the atomizer includes a mesh component at least partially surrounded by a piezoelectric element configured to vibrate the mesh component.

25. The diffuser of claim 19, further comprising circuitry configured to control operation of the diffuser; and
wherein the receptacle is removable from the diffuser and includes:
a memory configured to store at least one user setting associated with a user of the diffuser; and
communication circuitry configured to transmit the at least one user setting to the circuitry; and
wherein the circuitry is further configured to:
receive the at least one user setting from the communication circuitry of the receptacle; and
control operation of the diffuser based at least in part on the received at least one user setting.

26. The diffuser of claim 19, wherein the atomizer includes a piezoelectric element configured to vibrate the mesh component; and
wherein the diffuser further comprises circuitry configured to:
control a vibration frequency of the piezoelectric element based on a target diffusion distance for the atomized mixture of base liquid and oil.

27. The diffuser of claim 19, further comprising:
an air vent fluidly connecting at least one of the mixing chamber and the base liquid tank to an exterior of the diffuser, wherein the air vent relieves an air pressure in at least one of the mixing chamber and the base liquid tank.

28. The diffuser of claim 19, wherein the volume of the mixing chamber is within the range of four milliliters to eleven milliliters.

29. The diffuser of claim 19, further comprising:
at least one actuator configured to dispense a controlled amount of base liquid from the base liquid valve and to dispense a controlled amount of oil from the oil valve.

30. The diffuser of claim 19, further comprising:
an actuator configured to be rotatably moved between at least a first position and a second position; and
wherein, in the first position, the actuator contacts the base liquid valve to dispense base liquid from the base liquid tank, and in the second position, the actuator contacts the oil valve to dispense oil from the oil container.

31. The diffuser of claim 19, wherein the amount of oil dispensed from the oil valve is based, at least in part, on a predetermined viscosity of the mixture of base liquid and oil, and wherein the predetermined viscosity is based, at least in part, on at least one of a predetermined distance for diffusing the mixture of base liquid and oil from the atomizer and a volume of the mixing chamber.

32. The diffuser of claim 18, further comprising:
a channel between the base liquid valve and the mixing chamber, wherein an angle of the channel with respect to the gravitational direction is based, at least in part, on a drainage rate of the base liquid from the base liquid tank.

33. The diffuser of claim 19, further comprising:
circuitry configured to:
determine an end of an operating cycle of the diffuser; and
in response to determining the end of the operating cycle, control actuation of the base liquid valve to rinse the mixing chamber for a predetermined period of time or with a predetermined amount of base liquid.

34. The diffuser of claim 19, further comprising a first removable face plate on a face side of the diffuser that is interchangeable with a second removable face plate, wherein the atomizer is further configured atomize the mixture of base liquid and oil from the face side, and wherein at least one of the appearance and a lighting arrangement of the first removable face plate differs from the second removable face plate.

35. The diffuser of claim 19, further comprising:
circuitry configured to:
estimate at least one of an amount of oil dispensed from the oil container and an amount of base liquid dispensed from the base liquid tank; and
respectively calculate at least one of a remaining amount of oil in the oil container based, at least in part, on the estimated amount of oil dispensed, and a remaining amount of base liquid in the base liquid tank based, at least in part, on the estimated amount of water dispensed.

36. A diffuser, comprising:
a base liquid tank;
a receptacle configured to receive an oil container;
a mixing chamber;
a base liquid valve configured to dispense base liquid from the base liquid tank to the mixing chamber;
an oil valve configured to dispense oil from the oil container to the mixing chamber; and
an atomizer configured to atomize a mixture of base liquid and oil from the mixing chamber to an exterior of the diffuser, wherein the atomizer includes a mesh component at least partially surrounded by a piezoelectric element configured to vibrate the mesh component.

37. The diffuser of claim 36, wherein the atomizer is further configured to diffuse the mixture of base liquid and oil in a direction substantially perpendicular to a gravitational direction in which gravity acts on the diffuser when the diffuser is in an operating orientation.

38. The diffuser of claim 36, wherein a surface of the mesh component forms part of an exterior surface of the diffuser, and wherein the atomizer is further configured to diffuse the atomized liquid from at least a portion of the surface directly to air that is external to the diffuser.

39. The diffuser of claim 36, wherein the mesh component of the atomizer is removable and includes a first mesh pattern, and wherein the mesh component is interchangeable with a second mesh component that has a second mesh pattern that diffuses the atomized mixture of base liquid and oil in a different diffusion range than the first mesh pattern when operating in the atomizer.

40. The diffuser of claim 36, further comprising circuitry configured to:
control a vibration frequency of the piezoelectric element based on a target diffusion distance for the atomized mixture of base liquid and oil.

41. The diffuser of claim 36, further comprising:
an air vent fluidly connecting at least one of the mixing chamber and the base liquid tank to the exterior of the diffuser, wherein the air vent relieves an air pressure in at least one of the mixing chamber and the base liquid tank.

42. The diffuser of claim 36, wherein the volume of the mixing chamber is within the range of 4 milliliters to eleven milliliters.

43. The diffuser of claim 36, wherein an interior surface of the mesh component forms part of the mixing chamber.

44. The diffuser of claim 36, further comprising circuitry configured to control operation of the diffuser; and
wherein the receptacle is removable from the diffuser and includes:
a memory configured to store at least one user setting associated with a user of the diffuser; and
communication circuitry configured to transmit the at least one user setting to the circuitry; and
wherein the circuitry is further configured to:
receive the at least one user setting from the communication circuitry of the receptacle; and
control operation of the diffuser based at least in part on the received at least one user setting.

45. The diffuser of claim 36, further comprising:
at least one actuator configured to dispense a controlled amount of base liquid from the base liquid valve and to dispense a controlled amount of oil from the oil valve.

46. The diffuser of claim 36, further comprising:
an actuator configured to be rotatably moved between at least a first position and a second position; and
wherein, in the first position, the actuator contacts the base liquid valve to dispense base liquid from the base liquid tank, and in the second position, the actuator contacts the oil valve to dispense oil from the oil container.

47. The diffuser of claim 36, wherein the amount of oil dispensed from the oil valve is based, at least in part, on a predetermined viscosity of the mixture of base liquid and oil, and wherein the predetermined viscosity is based, at least in part, on at least one of a predetermined distance for diffusing the mixture of base liquid and oil from the atomizer and a volume of the mixing chamber.

48. The diffuser of claim 36, further comprising:
a channel between the base liquid valve and the mixing chamber, wherein an angle of the channel is based, at least in part, on a drainage rate of the base liquid from the base liquid tank.

49. The diffuser of claim 36, further comprising:
circuitry configured to:
determine an end of an operating cycle of the diffuser; and
in response to determining the end of the operating cycle, actuate the base liquid valve to rinse the mixing chamber for a predetermined period of time or with a predetermined amount of base liquid.

50. The diffuser of claim 36, further comprising a first removable face plate on a face side of the diffuser that is interchangeable with a second removable face plate, wherein the atomizer is further configured to atomize the mixture of base liquid and oil from the face side, and wherein at least one of the appearance and a lighting arrangement of the first removable face plate differs from the second removable face plate.
